(19) United States
(12) Patent Application Publication
(10) Pub. No.: US 2012/0094949 A1
(43) Pub. Date: Apr. 19, 2012

(54) HISTONE MODIFICATION PATTERNS FOR CLINICAL DIAGNOSIS AND PROGNOSIS OF CANCER

(75) Inventors: David W. Dawson, Pacific Palisades, CA (US); Siavash K. Kurdistani, Los Angeles, CA (US); David B. Seligson, Los Angeles, CA (US)

(73) Assignee: The Regents of the University of California, Oakland, CA (US)

(21) Appl. No.: 13/262,219

(22) PCT Filed: Apr. 14, 2010
(86) PCT No.: PCT/US10/31112

§ 371 Date: Dec. 27, 2011

Related U.S. Application Data
(60) Provisional application No. 61/169,212, filed on Apr. 14, 2009.
(60) Provisional application No. 61/169,216, filed on Apr. 14, 2009.
(60) Provisional application No. 61/225,162, filed on Jul. 13, 2009.

(51) Int. Cl. A61K 31/7068; C40B 30/04; A61P 35/00; G01N 33/566

(52) U.S. Cl. 514/49; 435/7.1; 506/9

(57) ABSTRACT

The present invention provides methods of diagnosing and providing a prognosis and therapy for cancer including, but not limited to, pancreatic cancer and responsiveness to thymidylate synthase inhibitor (e.g., 5-FU) therapy, by identifying cancers with altered histone modification patterns selected from the group consisting of H3K4me2, H3K9me2, or H3K18ac.

### CROSS-REFERENCES TO RELATED APPLICATIONS

This application claims priority benefit of U.S. Provisional Application Ser. No. 61/169,212, filed on Apr. 14, 2009, U.S. Provisional Applications Ser. No. 61/169,216, filed Apr. 14, 2009, and U.S. Provisional Application Ser. No. filed 61/225,162, filed on Jul. 13, 2009, the contents of which are incorporated herein in their entireties.

### STATEMENT AS TO RIGHTS TO INVENTIONS MADE UNDER FEDERALLY SPONSORED RESEARCH AND DEVELOPMENT

This research was supported in part by the Government under a grant from the National Cancer Institute Early Detection Research Network (EDRN NCI CA-86366) and also from by grants from the Hirschberg Foundation for Pancreatic Cancer Research, CURE Digestive Diseases Research Center (DK041301, NIH/NIDDK) and Radiation Therapy Oncology Group Translational Research Program funded by NCI U10CA21661; the Government has certain rights in this invention.

### REFERENCE TO A “SEQUENCE LISTING,” A TABLE, OR A COMPUTER PROGRAM LISTING APPENDIX SUBMITTED ON A COMPACT DISK

NOT APPLICABLE

### FIELD OF THE INVENTION

This invention relates to the use of global histone modifications to predict the prognosis of cancers and to predict the likelihood that a patient would respond to therapy with a thymidylate synthase inhibitor.

### BACKGROUND OF THE INVENTION

Pancreatic adenocarcinoma is a highly aggressive and lethal cancer for which there are limited therapeutic options. Along with genetic events, tumor-associated epigenetic alterations are important determinants in the initiation and progression of pancreatic cancer (Maitra, A., Hruban, R. H., Annu Rev Pathol 3:157-88 (2008); Hezel et al., Genes Dev 20:1218-49 (2006)) and represent promising biomarkers and therapeutic targets. Epigenetic alterations in cancer include genome-wide and locus-specific changes in DNA methylation and post-translational histone modifications, which influence chromatin accessibility and gene activity (Bernstein et al., Cell 128:669-81 (2007); Ting et al., Genes Dev 20:3215-3231 (2006); Esteller, M., Nat Rev Genet 8:286-98 (2007)).

Locus-specific changes in histone acetylation or methylation have been linked to the altered expression of several critical genes in pancreatic cancer (Fitzgerald et al., Neoplasia 5:427-36 (2003); Fujii et al., J Biol Chem 283:17324-32 (2008); Kikuchi et al., Oncogene 21:2741-9 (2002); Kumagai et al., Int J Cancer 124:827-33 (2009)), while widespread changes in gene expression seen on microarrays after treatment of cell lines with histone deacetylase inhibitors suggest that histone modifications may play a much broader role in regulating gene expression in pancreatic cancer (Kumagai et al., Int J Cancer 124:827-33 (2009); Sato et al., Cancer Res 63:3735-42 (2003)).

Cancer-associated genome-wide alterations in histone modifications include changes in their levels and distribution across the genome, such as at gene promoters, repetitive DNA sequences and other heterochromatin regions (Esteller, M., Nat Rev Genet 8:286-98 (2007)). Finally, heterogeneity in cellular levels of histone modifications across a given tumor as demonstrated by cell-to-cell differences in immunohistochemical staining of tumor cell nuclei (Kurdistani, S. K., Br J Cancer 97:1-5 (2007)) adds a further layer of complexity to the spectrum of changes that typify the cancer epigenome.

Aberrations in histone modifications occur in human disease, including cancer. Aberrations in post-translational modifications of histones have been shown to occur in cancer cells but only at individual promoters (Jacobson, et al., Curr. Opin. Genet. Dev. 9:175-84 (1999)) and have not been related to clinical outcome. These aberrations may occur locally at promoters by inappropriate targeting of histone modifying enzymes, leading to improper expression or repression of individual genes that play important roles in tumorigenesis. However, despite a large number of genes examined, little similarity in local, gene-targeted histone modification changes in different cancers is reported. Aberrant modification of histones associated with DNA repetitive sequences has also been reported. These aberrations include lower levels of histone H4 K16Ac and K20diMe in hematological malignancies and colorectal adenocarcinomas. None of these changes, either at individual genes or at repetitive DNA elements, however, has been related to clinical outcome.

Histone modifications, such as acetylation and methylation of lysines (K) and arginines (R), which also occur over large regions of chromatin including non-promoter sequences, are referred to as global histone modifications (Vogelauer, et al., Nature 408:495-8 (2000)). As noted above, enzymes that modify histones exhibit altered activity in cancer. For instance, missense mutations of p300 histone acetyltransferases and loss of heterozygosity at the p300 locus are associated with colorectal and breast cancers, and glioblastomas (Giles, et al., Trends. Genet. 14:178-83 (1998); Gayther, et al., Nat. Genet. 24:300-3 (2000); Muraoka, et al., Oncogene 12:1565-9 (1996)). The consequence of the altered activity of histone-modifying enzymes has so far been linked to inappropriate expression of few genes that may play a role in tumor biology. For instance, p300 is involved in androgen receptor transactivation, potentially playing an important role in progression of prostate cancer (Debes, et al. Cancer Res. 63:7638-40 (2003))“.

However, in addition to being targeted to promoters, these enzymes also affect most nucleosomes throughout the genome independently of apparent sequence-specific DNA binding proteins (Vogelauer, et al., Nature 408:495-8 (2000); Reid, et al., Mol. Cell 6, 1297-307 (2000); Krebs, et al., Cell 102, 587-98 (2000)). Furthermore, the histone modifying enzymes possess a high degree of substrate specificity which differentiate between the histone sub-types as well as individual side-chains within each histone (Peterson, et al., Curr. Biol. 14, R546-51 (2004); Suka, et al., Mol. Cell 8:473-9 (2001)). Thus, individual residues will be modified globally to varying extent, reflecting the selective but widespread activity of the histone-modifying enzymes.

There is a need for improved markers for cancer prognosis and therapy. The present invention meets these needs and relates to our surprising discovery that specific histone modifications are useful prognostic and predictive biomarkers in pancreatic and other cancers. These cellular levels of histone modifications define previously unrecognized subsets of pancreatic adenocarcinoma patients with distinct epigenetic phenotypes and clinical outcomes and represent prognostic and predictive biomarkers that also inform clinical decisions including the use of 5-FU and similar chemotherapies.

### BRIEF SUMMARY OF THE INVENTION

In one aspect, the present invention provides methods of providing a prognosis for a human subject with cancer including, but not limited to, pancreatic cancer. The methods generally comprise contacting a test tissue sample from an individual having the cancer; and detecting one, two or more histone protein modifications selected from H3K4me2, H3K9me2, or H3K18ac in the test tissue sample and comparing them to values representative of patients classified according to their survival history. Typically, the tissue sample is a tissue biopsy. As lower levels of histone modifications H3K4me2, H3K9me2, or H3K18ac are significantly associated with reduced survival in cancer patients, the presence of a similar low level of a modification in the for an individual leads to a prognosis of reduced survival time or life expectancy. Conversely, the presence of a higher level of a modification leads to a prognosis of an increased survival time or life expectancy. In preferred embodiments, the individual has less advanced disease (i.e. low grade or stage), the category in which prognostic markers are acutely needed.

In another aspect the invention provides methods of treating an individual having a low grade cancer including, but not limited to, a pancreatic cancer, said method comprising the step of determining the global histone modification level in the test tissue sample in comparison to a comparison tissue sample (persons with a known survival, therapeutic or disease outcome) and administering a more aggressive cancer therapy than usual for the grade to the patient when the histone modification level indicates that the cancer is likely to progress in severity or metastasize based upon the comparison. In some embodiments, the steps include obtaining a test or biopsy sample from the individual and contacting the test or biopsy tissue sample from the individual with an antibody or aptamer that specifically binds to a modified histone protein selected from H3K4me2, H3K9me2, and H3K18ac; and

In another aspect the invention provides methods of assessing the response of a cancer patient including, but not limited to, pancreatic cancer patients, to a medical treatment, comprising the step of determining the histone modification level in the test tissue sample in comparison to a tissue sample taken from the patient before the treatment, or earlier or later in the course of a treatment, or before and after a treatment has been modified. In some embodiments, the therapy is immunotherapy, targeted molecular therapy, epigenetic therapy, chemotherapy or radiation or a pro-apoptosis therapy. In some embodiments, a test or biopsy sample is obtained from the patient and the sample is contacted with an antibody that specifically binds to a modified histone protein selected from H3K4me2, H3K9me2, and H3K18ac.

In another aspect the invention provides a kit comprising at least two antibodies which each bind a different histone protein modification. In some embodiments, the antibodies are selected from the group consisting of H3K4me2, H3K9me2, or H3K18ac. In some embodiments, the antibodies are labeled with a detectable moiety. In some embodiments, the kits provide reagents for detecting these antibodies when used as markers. In some embodiments, the kits provide additional reagents and/or instructions for immunohistochemical staining of tissues using the antibodies. In some embodiments, the kits further comprise instructions on how to assess the resulting immunohistochemical staining with respect to cancer risk or prognosis.

Accordingly, the invention provides a method for giving a prognosis to, or for, a subject having cancer including, but not limited to, pancreatic cancer, said method comprising determining the histone modification level for H3K4me2, H3K9me2, or H3K18ac in a tissue sample from the cancer, wherein the presence of a low level of the histone modification indicates a poorer prognosis for survival and the presence of a high histone modification level for H3K4me2, H3K9me2, or H3K18ac indicates a better prognosis for survival. In some embodiments, the subject has node-negative cancer or is receiving 5-fluorouracil. In other embodiments of any of the above, a positive tumor cell staining of the histone modifications H3K4me2, H3K9me2, or H3K18ac is used to classify the patient as low or high staining, wherein a low staining classification supports a prognosis of a poorer overall survival. In still other embodiments, the prognosis is based upon low histone modification levels of both H3K4me2 and H3K18ac (the worst prognosis grouping is defined as low levels of either one or both of the modifications). In some embodiments, a low histone modification level for both H3K4me2 and H3K18ac predicts a lower likelihood of survival. In preferred embodiments, the histone modification levels are determined by immunocytochemistry. The subjects may be classified into high or low risk groups by the percent rank staining of a histone modification selected from H3K4me2, H3K9me2, and H3K18ac. For instance, a H3K9dime ≧10%, >60% for H3K4me2 or >35 percentile staining K18ac.

In another aspect, the invention provides a means for predicting the response of a subject having pancreatic cancer, or another cancer for which 5-FU or another thymidylate synthase inhibitor is a treatment with or without Leucovorin, to 5-FU or the thymidylate synthesase inhibitor therapy (e.g. raltitrexed, pemetrexed, nolatrexed, ZD9331, and GS7904L) wherein the prediction is based upon the presence or absence of lower level of H3K4me2 or H3K18ac as compared to values determined for comparison populations for whom the response is known. In the method, a lower level of the modification predicts a worse-disease free survival. The comparison groups can be dichotomous, continuous, or discretely graded with respect to modification levels and their associated survival outcomes. Cancers for which 5-FU is a treatment include, but are not limited to, colon, rectal, head and neck, breast, ovarian cancer, and basal cell cancer of the skin. In most cases 5-FU is used in combination with Leucovorin.

In still another aspect the invention provides a method of identifying a patient having pancreatic cancer patient or a patient having a cancer for which 5-fluorouracil or another thymidylate synthase inhibitor is utilized as a standard chemotherapy for whom the addition of a histone deacetylase inhibitor to 5-FU would be beneficial. In the method, the level of the H3K18ac histone modification is determined in a tissue sample from the cancer of the patient. A low level of the modification (based upon a similarity to values determined for comparison populations for whom the modifications and response profile to 5-FU without the inhibitor were known) being indicative that a histone deacetylase inhibitor would be beneficial as a therapy or additionally be beneficial. Accordingly, the invention also provides methods of treatment wherein a patient who is so identified is then treated with the inhibitor and, optionally, with 5-FU or another treatment described herein.

In still another aspect the invention provides a method of identifying a patient having a cancer for which 5-fluorouracil is utilized as a standard chemotherapy (e.g., colorectal cancer, breast cancer) for whom the addition of a histone deacetylase inhibitor to 5-FU therapy would be beneficial. In the method, the level of the H3K18ac histone modification is determined in a tissue sample from the cancer of the patient. A low level of the modification (based upon a similarity to values determined for comparison populations for whom the modifications and response profile to 5-FU without the inhibitor were known) being indicative that a histone deacetylase inhibitor would additionally be beneficial. Accordingly, the invention also provides methods of treatment wherein a patient who is so identified is then treated with 5-FU and the inhibitor.

In further embodiments of any of the above aspects providing a prognosis, identification, assessment, treatment, prediction or determination, a positive tumor cell staining of the histone modifications H3K4me2, H3K9me2, or H3K18ac is used to classify the patient as low or high staining, wherein a low staining classification supports a prognosis of a poorer overall survival and/or thymidylate inhibitor non-responsiveness. In still other embodiments, the prognosis is based upon low histone modification levels of both H3K4me2 and H3K18ac (the worse prognosis grouping being defined as low levels of either one or both of these modifications). In some embodiments, a low histone modification level for both H3K4me2 and H3K18ac predicts a lower likelihood of survival. In preferred embodiments, the histone modification levels are determined by immunocytochemistry. The subjects may be classified into high or low risk groups by the percent rank staining of a histone modification selected from H3K4me2, H3K9me2, and H3K18ac. In some embodiments, the invention provides for the selection of a more aggressive therapy for a patient identified as being resistant to therapy or likely to have a worser outcome or prognosis (e.g., poorer overall survival) based upon the histone modification pattern.

In some embodiments of any of the above aspects, the histone modifications levels for one, two or three of the histone modifications selected from H3K4me2, H3K9me2, and H3K18ac are used to provide the prognosis, identification, assessment, treatment, prediction or determination. In such embodiments where only two modifications are selected, the histone modifications may be selected from the group consisting of H3K4me2 and H3K9me2, H3K4me2 and H3K18ac, or H3K9me2, and H3K18ac. In some embodiments, the classification as to whether a modification is low or high is based upon the histone rule. The comparison groups can be dichotomous, continuous, or discretely graded with respect to modification levels and their associated survival outcomes.

In any of the above embodiments for any of the above aspects, the patient can be human and the cancer an adenocarcinoma, a pancreatic cancer, a breast cancer, a prostate cancer, a lung cancer, a breast cancer, a colon cancer, a rectum cancer, an esophageal cancer, a gallbladder or kidney cancer. In some embodiments of any of the above, the methods provide additional non-redundant prognostic information useful in providing a prognosis or selecting a therapy for a cancer.

In some embodiments, each tumor is assigned into a low or high level staining group based on its percent rank based upon the median percent of cells staining positive to its , including H3K4me2 (<60 vs. ≧60 percent rank), H3K9me2 (<30 vs. ≧30 percent rank for the RTOG TMA or <25 vs. ≧25 percent rank for the UCLA Stage I/II TMA) and H3K18ac (<35 vs. ≧35 percent rank).

### BRIEF DESCRIPTION OF THE DRAWINGS

FIG. 1. Cellular heterogeneity of histone modifications in pancreatic adenocarcinoma. (A) Representative immunohistochemistry for histone modifications at 10× or 40× (inset) objective from tumors of either low (patient 1) or high (patient 2) grade histology. The distribution of tumors showing indicated percentage of tumor cells with positive nuclear staining is shown for each histone modification in the (B) RTOG 9704 or (C) UCLA Stage I/II TMA.

FIG. 2. Overall patient survival in the UCLA Stage I/II pancreatic cancer TMA based on indicated histone modification group. Kaplan-Meier plots visualize survival probabilities for the high (solid line) versus low (dashed line) level histone group for (A) H3K4me2, (B) H3K18ac, (C) H3K9me2 and (D) low H3K4me2 and/or H3K18ac versus high H3K4me2 and H3K18ac. p-values for Log rank tests.

FIG. 3. Overall survival in RTOG 9704 TMA for indicated histone modification after first stratifying on treatment arms. Patients were stratified based on adjuvant chemotherapy (A-B, 5-fluorouracil or C-D, gemcitabine). Kaplan-Meier plots were then used to visualize survival probabilities for patients with either high (solid line) versus low (dashed line) levels of (A, C) H3K4me2 or (B, D) H3K9me2. p-values for Log rank tests.

FIG. 4. Cellular heterogeneity in levels of histone modifications in primary cancer tissues. Immunohistochemical staining of cancer tissues from (A) lung adenocarcinoma (grade 2) and (B) kidney clear cell carcinoma (grade 1) with an anti-H3K18ac antibody. Percentage of cancer cells with brown nuclei determines the global levels of each histone modification for a given individual. Magnification: 10×, left panel; 40×, right panel. Distribution of patients for the levels of H3K4me2 (black bars) and H3K18ac (grey bars) in cancer tissues from (C) lung and (D) kidney are shown. The graphs represent the fraction of patients (y-axis) with indicated levels of histone modifications as percent cell staining (x-axis).

FIG. 5. Prediction of clinical outcome in different carcinomas by histone modifications. For each cancer type, patients were first assigned to two groups based on the levels of H3K4me2 and H3K18ac, and then, their clinical outcomes were compared. Kaplan-Meir plots are used to visualize survival probabilities of the two groups (Group 1, black line; Group2, red line) in (A) lung (Log rank p=0.018, n=159) and (B) kidney (Log rank p=0.028, n=192). Tabulated in the inset boxes is the distribution of the patients in each group according to grade.

FIG. 6. The cellular levels of H3K9me2 predict clinical outcome in prostate and kidney cancers. Distribution of patients for the levels of H3K9me2 in cancer tissues from (A) prostate and (C) kidney are shown. The graphs represent the fraction of patients (y-axis) with indicated levels of histone modifications as percent cell staining (x-axis). For each cancer type, patients were first assigned to two groups based on the levels of H3K9me2, and then, their clinical outcomes were compared (Group 1, H3K9me2>10%, black line; Group2, H3K9me2≦10%, red line). Kaplan-Meir plots are used to visualize the difference in outcome of the two groups in (B) low grade prostate (Log rank p=0.0043, n=109) and (D) all kidney (Log rank p=0.00092, n=359) cancer patients. Tabulated in the inset boxes is the distribution of the patients in each group according to grade.

FIG. 7. Cellular heterogeneity in levels of histone modifications in cancer cell lines. (A) Immunohistochemical examination of H3K9me2 in LNCaP and PC3 prostate cancer cell lines. Note the increased percentage of PC3 cells with lower levels of H3K9me2 (blue nuclei) compared to LNCaP cells. (B) Western blot of acid-extracted histones from LNCaP and PC3 cells for H3K9me2 levels and histone H3 (irrespective of modifications) as a loading control. The triangles indicate increased loading from left to right.

FIG. 8. Global levels of H3K9me2 correlates with its levels at repetitive DNA elements. (A) ChIP-chip analysis of H3K9me2 in LNCaP and PC3 cells. Each row represents the region from −5.5 to +2.5 of annotated transcription start site (TSS) for a given gene which is divided into 16 fragments of 500-bp each. Genes are grouped based on similarity of e1a-binding pattern across the 8 kb promoter region. The colors indicate relative enrichment or depletion of ChIPed DNA (yellow) vs. input (blue) from each cell. (B) Correlations of H3K9me2 levels at each of the 16 fragments across all promoters between LNCaP and PC3 cells. (C) ChIP-quantitative real-time PCR analyses of the levels of H3K9me2 and H3K18ac at the indicated DNA repetitive elements. The values are represented as percentage of input. The error bars represent standard deviation of 3 independent experiments. Histone H3 ChIP was used as a control to show that lower modification levels in PC3 cells are not due to nucleosome loss.

FIG. 9. Cellular patterns of histone modifications in kidney cancer. (A) The cellular histone modification patterns based on H3K4me2 and H3K18ac did not predict outcome in patients with metastatic disease (p=0.99, n=163). (B) Kidney cancer patients in the low grade categories (grades 1 and 2, n=221) were assigned to two groups based on the levels of H3K4me2 and H3K18ac, and their clinical outcomes were compared. A Kaplan-Meir plot is used to visualize survival probabilities of the two groups (Group 1, black line; Group 2, red line) (Log rank p=0.0055, HR=1.9, 95% CI 1.2-3.1). The histone modifications did not predict outcome in patients with grades 3 and 4 kidney cancer (data not shown).

FIG. 10. Cellular patterns of H3K9me2 predict prognosis kidney cancer. Tumors were first stratified based on tumor localization (localized vs. metastatic disease). Patients in each stratum were assigned to two groups based on the levels of H3K9me2—≦10% staining, Group 2, red and blue lines; >10% staining, Group 1, black and green lines—and their clinical outcomes were compared. A Kaplan-Meir plot is used to visualize survival probabilities of the two H3K9me2 groups in each stratum. In both localized (black and red lines) and metastatic disease (green and blue lines), lower levels of H3K9me2 predicted poorer survival probabilities.

FIG. 11. Cellular heterogeneity in levels of histone modifications in cancer cell lines. (A) Immunohistochemical examination of H3K4me2 and H3K18ac in LNCaP and PC3 prostate cancer cell lines. Note the increased percentage of PC3 cells with lower levels of histone modifications (blue nuclei indicated by orange arrows) compared to LNCaP cells. The intensity of staining is also (B) Western blot of acid-extracted histones from LNCaP and PC3 cells for H3K4me2 and H3K18ac levels. The triangles indicate increased loading from left to right.

FIG. 12. Histone modifications predict prognosis in breast cancer.

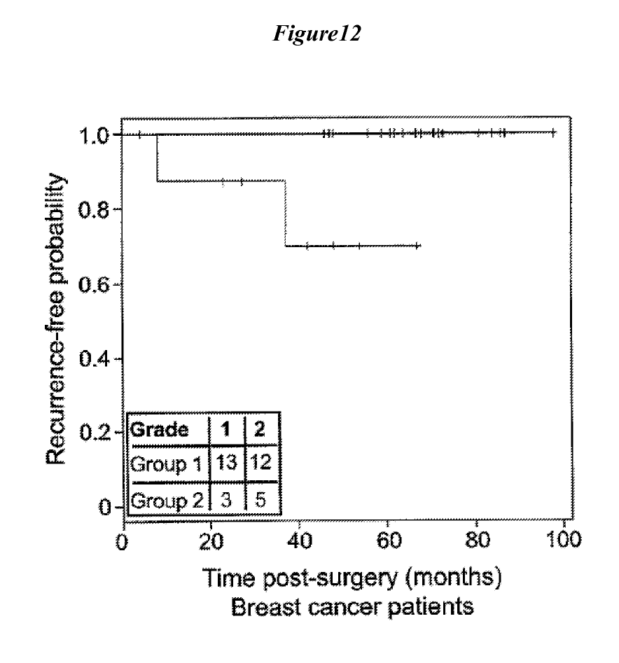

### DETAILED DESCRIPTION

Cellular patterns of histone modifications provide additional independent prognostic information for several tumor types, including prostate (Seligson et al., Am J Pathol 174:1619-1628 (2009); Seligson et al., Nature 435:1262-6 (2005)), kidney (Seligson et al., Am J Pathol 174:1619-1628 (2009)), lung (Seligson et al., Am J Pathol 174:1619-1628 (2009); Seligson et al., Nature 435:1262-6 (2005); Barlesi et al., J Clin Oncol 25:4358-64 (2007)), gastric (Park et al., Ann Surg Oncol 15:1968-76 (2008)) and ovarian cancer (Wei et al., Mol Carcinog 47:701-6 (2008)). Low cellular levels of H3K27me3 were also recently shown to be associated with poor outcome in pancreatic cancer (Wei et al., Mol Carcinog 47:701-6 (2008)). However, cellular levels of histone modifications have not been shown to predict response to a specific therapy. Using tissue microarrays from two large pancreatic adenocarcinoma patient cohorts, we examined the cellular levels of three histone modifications not previously studied in pancreatic cancer, including H3K4me2, H3K9me2 and H3K18ac. We found these modifications to be highly significant and independent prognostic factors in pancreatic cancer. In addition, we found that lower cellular levels of H3K4me2 and H3K9me2 predicted worse survival outcome specifically for patients receiving adjuvant 5-FU chemotherapy. Our data indicate that cellular levels of histone modifications represent novel prognostic markers for pancreatic cancer and are helpful in predicting response to 5-FU.

Here, we have demonstrated that low cellular histone modification levels identify pancreatic cancer patients less likely to derive survival benefit from adjuvant 5-FU chemotherapy, while high cellular histone levels identify patients who derive similar survival benefit from the use of either adjuvant gemcitabine or 5-FU chemotherapy. We conclude that cellular histone modification levels represent a novel category of biomarkers able to predict response to adjuvant 5-fluorouracil chemotherapy in resected pancreatic cancer, and with potential applicability to the neoadjuvant setting or advanced pancreatic cancer. More generally, cellular histone modification levels can prove to be useful predictive biomarkers for response to 5-FU or other thymidylate synthase inhibitors in other malignancies (i.e., colorectal or breast cancer) where 5-fluorouracil is utilized as a standard chemotherapy.

In another aspect the invention provides methods of treating an individual having a low grade or stage of cancer, by determining whether the individual has a low grade cancer and by contacting a test tissue sample from the individual with an antibody that specifically binds to a modified histone protein selected from H3K4me2, H3K9me2, and H3K18ac; and determining the global histone modification pattern in the test tissue sample in comparison to a control tissue sample and administering a more aggressive cancer therapy to the patient when the global histone modification pattern indicates that the cancer is likely to progress or metastasize. The determining the grade or stage of the cancer can be before or after the histone protein modification pattern is determined.

In yet other embodiments, the invention provides a method of targeting patients for more aggressive or alternative cancer therapy or increased surveillance for a cancer recurrence based upon an altered global histone modification pattern in a tissue sample from the patient taken before, during, or after surgical removal of the cancerous tissue before, during, or after another cancer treatment. The altered global histone modification pattern can be determined as described herein. Patients identified as having altered an global histone modification pattern(s) selected from H3K4me2, H3K9me2, and H3K18ac with an increased risk of metastasis, recurrence or a therapy resistant cancer can be further selected on that basis for treatment with immunotherapy, chemotherapy and/or radiation.

In another aspect the invention provides methods of assessing the response of a cancer patient to a medical treatment, comprising the steps of contacting a test tissue sample from the individual receiving the treatment with an antibody that specifically binds to a modified histone protein selected from H3K4me2, H3K9me2, and H3K18ac; and determining the global histone modification pattern of selected from H3K4me2, H3K9me2, and H3K18ac; in the test tissue sample in comparison to a tissue sample taken from the patient before the treatment, or earlier or later in the course of a treatment, or before and after a treatment has been modified. In some embodiments, the therapy is hormonal ablation therapy or chemotherapy or radiation or a pro-apoptosis therapy.

In another aspect, the invention provides a method of providing a prognosis for a cancer by contacting a test tissue sample from an individual at risk for or known to have a cancer with an antibody that specifically binds to a modified histone protein; and determining the global histone modification pattern in the test tissue sample in comparison to a control tissue sample; thereby providing a prognosis for said cancer by identification of an altered global histone modification pattern. In some embodiments, the tissue sample is a tumor biopsy sample. In some embodiments, the cancer or tumor is prostate, bladder, kidney, colon or breast cancer. In preferred embodiments, the individual has less advanced disease (i.e. low grade or stage), the category in which prognostic markers are acutely needed.

In another aspect, the invention provides kits comprising at least two antibodies which each bind a different histone protein modification. In some embodiments, the antibodies are selected from the group consisting of H3 K9 acetylation, H3 K18 acetylation, H4 K12 acetylation, H3 K4 dimethylation, H3 K9diMe, and H4 R3 dimethylation. In some embodiments, the antibodies are labeled with a detectable moiety. In some embodiments, the kits provide reagents for detecting the antibody when bound to a histone protein having the histone protein modification recognized by the antibody. In other embodiments, the kits have instructions relating altered histone modification patterns to an increased or decreased risk of cancer metastasis or progression. In other embodiments, the kits further comprise reagents for use in immunohistochemical methods using the antibodies.

In further embodiments of any of the above aspects, the global histone protein modification is selected from one or more of the group consisting of H3 K9 acetylation, H3 K18 acetylation, H4 K12 acetylation, H3 K4 dimethylation, H3 K9dimethylation, and H4 R3 dimethylation. In still further embodiments of such, the cancer or tumor is prostate, bladder, kidney, colon or breast cancer. The cancer can be a metastatic cancer.

In some embodiments of any of the above aspects, the global histone modification pattern of one, two, three, four, or at least two or three different histone protein modifications is detected. In further embodiments, the at least two different histone protein modifications are selected from the group consisting of H3 K9 acetylation, H3 K18 acetylation, H4 K12 acetylation, H3 K4 dimethylation, a H3 K9dimethylation, and H4 R3 dimethylation. In some preferred embodiments, the histone protein modifications are H3 K4 dimethylation and H3K18 acetylation. In other embodiments, the histone proteins are selected from methylations and acetylations of either or both H3 and H4 histone proteins. In other embodiments, the histone proteins are selected from methylations and acetylations of either or both H2A and H2B histone proteins. The histone proteins and individual are preferably human.

In some embodiments, the altered global histone modification pattern in the individual who has cancer or is suspected of having a cancer is determined by (a) obtaining a tissue sample from a portion the subject wherein the portion has or is suspected of having cancer cells therein; and (b) detecting one, two, three, four or more global histone modifications in the sample to provide a global histone modification pattern and (c) comparing the histone modification pattern to a control or normal global histone modification pattern for a subject to identify an altered global histone modification pattern. In further such embodiments, the global histone modifications are detected using antibodies which specifically bind the histone protein modification of interest. The antibody may be a monoclonal antibody or a polyclonal antibody directed toward the histone modification pattern of interest. In some embodiments, the method further comprises the step of fixing the cells and detecting the global histone modifications in the fixed cells.

In further such embodiments, and in any of the above aspects generally, the immunohistochemical staining uses antibodies to specifically bind the histone protein modification of interest. The antibody may be a monoclonal antibody or a polyclonal antibody directed toward the histone modification pattern of interest. The antibody may be labeled with a detectable label (e.g., a radioactive label, and enzymatic label, a fluorescent label, or chemiluminescent label, or a molecular tag). The label bound to the histone modification of interest may be detected by autoradiography, fluorimetry, luminometry, or phosphoimge analysis. In a preferred embodiment, the global histone modifications are detected for a plurality of individual fixed cells in the sample and the intensity and/or frequency of immunohistochemical staining is determined for each of the plurality such that a frequency distribution of cells according to staining intensities are obtained over an area of interest. Preferably, the area of interest focuses on cells having an altered phenotype suggestive of a cancer. The area may be defined empirically according to the region of the sample having the most intense staining (if a modification positively correlates with the risk, grade, or progression of cancer) or the least intense staining if the modification negatively correlates with the risk, grade, or progression of cancer. The area may be of a predetermined size sufficient to provide a valid measure of staining patterns in the area of interest. Multiple areas may be sampled and compared from each of the tissue samples.

In some embodiments of any of the above aspects, the histone protein modification is selected from one or more of the group consisting of H3 K9 acetylation, H3 K18 acetylation, H4 K12 acetylation, H3 K4 dimethylation, H3 K9dimethylation, and H4 R3 dimethylation. In still further embodiments of such, the cancer or tumor is prostate, bladder, kidney, colon or breast cancer. The cancer can be a metastatic cancer. In some embodiments, the cut-off for a high or level of the modification claim for the histone modification is about ≧10% for H3K9dime, about >60% for H3K4me2 or about >35 percentile staining H3K18ac.

In some embodiments of any of the above aspects, the global histone modification pattern of at least two or three different histone protein modifications is detected. In further embodiments, the at least two different histone protein modifications are selected from the group consisting of H3 K9 acetylation, H3 K18 acetylation, H4 K12 acetylation, H3 K4 dimethylation, H3 K9dimethylation, and H4 R3 dimethylation. In some preferred embodiments, the histone protein modifications are H3 K4 dimethylation and H3K18 acetylation. In other embodiments, the histone proteins are selected from methylations and acetylations of either or both H3 and H4 histone proteins. The histone proteins are preferably human. In some embodiments, the selected modifications are modifications of H3 or H4. In some further embodiments, the selected modifications are methylations and/or acetylations of H3 or H4. In other embodiments, the selected modifications comprise a phosphorylation or ubiquinylation of H3 or H4. In further embodiments, the at least two different histone protein modifications are selected from the group consisting of H3 K9 acetylation, H3 K18 acetylation, H4 K12 acetylation, H3 K4 methylation(s), H3 K9 methylation (s), and H4 R3 methylation(s). Characterization of the global histone modification pattern allows the altered global histone modifications which are of diagnostic and prognostic value to be determined.

In other embodiments of any of the above aspects and embodiments, the methods use antibodies which specifically bind to the histone protein modification of interest to detect the modifications. The antibody may be a monoclonal antibody or a polyclonal antibody directed toward the histone modification pattern of interest. In some embodiments, a plurality of global histone modification patterns are determined for the sample. The histones to be analyzed for particular modifications may be first isolated from the sample and detected using immunochemical methods in a fluid medium.

In some embodiments, the ratio of a modified histone protein to the total levels of the histone protein provide a predictive measure based upon altered global histone modification patterns. For instance, a sample may be analyzed using an antibody which detects modified and unmodified forms of a histone protein and an antibody which selectively detects histones have the modification of interest. The ratio of the two in a population of cells or in a sample is determined and is compared to the ratio for a normal cell to establish a predictive ratio of altered global histone protein modification which can be used in the methods according to the invention.

In some embodiments, the altered global histone modification patterns are predictive of whether a cancer or tumor will be refractory to treatment or therapy resistant, or provide a better prognosis (e.g., increased likelihood of survival (e.g., survival at 6 months, 1 year, 2, years, 3, years, 4 years, 5 years or longer), or decreased likelihood of the recurrence of the cancer, or a decreased likelihood of the metastasis of the cancer; or the likelihood of a positive response to therapy with a thymidylate synthase inhibiton including, but not limited to 5-FU).

In some embodiments of any of the above, the tissue is disaggregated by enzymatic, grinding, or other means and the global histone modification patterns of individual cells are characterized by immunofluorescence staining using the antibodies described herein followed by FACS sorting and/or scoring and counting of the cells which can provide a frequency distribution of the global histone modification frequencies for the sample. In such methods it can also be useful to employ other fluorescent markers identifying the particular cell or its phenotype to facilitate in the sorting and counting of the particular cells of interest.

This present invention relates to our discovery that changes in global levels of individual histone modifications are associated with the presence of cancer and, importantly, are predictive of clinical outcome (see, WO 2006/119264 and U.S. Patent Application Publication No. US20080248039, corresponding to U.S. patent application Ser. No. 11/912,429 filed May 29, 2008, which are assigned to the same assignee as the present invention and which are incorporated herein by reference in their entireties). Through immunohistochemical staining of primary prostatectomy samples, the percentage of cells that stain for histone acetylation (Ac) and di-methylation (diMe) of five residues in histones H3 and H4 was determined. Grouping of samples with similar patterns of modifications identified two disease sub-types with distinct risks of tumor recurrence among patients with low-grade prostate cancer. These histone modification patterns were predictors of outcome independent of tumor stage, pre-operative prostate-specific antigen (PSA) levels, and capsule invasion. Thus, widespread changes in specific histone modifications represent novel molecular heterogeneity in prostate cancer, and underlie the broad range of clinical behavior displayed by cancer patients. In subsequent work, the markers were further identified to be useful markers with respect to lung cancer, kidney cancer, breast cancer, colon cancer, and other cancers, as well as prostate cancer.

This evidence indicates that changes in bulk or global histone modifications of cancer cells is predictive of clinical outcome. The mechanistic basis of such changes are currently unclear but maybe related to the altered expression and/or global activities of various histone modifying enzymes. In combinations of two or more, these changes proved to be particularly indicative of risk of tumor recurrence in patients, in particular in patients with low-grade prostate cancer. Considering the substantial number of modifications on histones, information on global patterns of other modification sites would help with further classification of all patients including those in the high-grade category. The utility of immunohistochemistry combined with availability of extensive set of antibodies to probe histone modifications, facilitates the application of this approach to other tumors and other histone modification patterns.

Accordingly, in one aspect, the invention provides a method of diagnosing a cancer by contacting a test tissue sample from an individual at risk of having a cancer or suspected of having cancer with an antibody that specifically binds to a modified histone protein; and determining the global histone modification pattern in the test tissue sample in comparison to a control tissue sample; thereby diagnosing said cancer by identification of an altered global histone modification pattern. In some embodiments, the tissue sample is a tumor biopsy sample. In some embodiments, the cancer or tumor is prostate, bladder, kidney, colon or breast cancer. In preferred embodiments, the individual has less advanced disease (i.e. low grade or stage), the category in which diagnostic markers are acutely needed. The global histone modification pattern can be scored according to standard immunohistochemical methodologies.

In another aspect the invention provides methods of treating an individual having a low grade or stage of cancer, by determining whether the individual has a low grade cancer and by contacting a test tissue sample from the individual with an antibody that specifically binds to a modified histone protein; and determining the global histone modification pattern in the test tissue sample in comparison to a control tissue sample and administering a more aggressive cancer therapy to the patient when the global histone modification pattern indicates that the cancer is likely to progress or metastasize. The determining the grade or stage of the cancer can before or after the histone protein modification pattern is determined.

In yet other embodiments, the invention provides a method of targeting patients for more aggressive or alternative cancer therapy or increased surveillance for a cancer recurrence based upon an altered global histone modification pattern in a tissue sample from the patient taken before, during, or after surgical removal of the cancerous tissue (e.g., prostectomy) or before, during, or after another cancer treatment. The altered global histone modification pattern can be determined as described herein. The cancer can be, for instance, a prostate cancer, ovarian cancer, renal cancer, lung cancer, breast cancer, colon cancer, leukemia, non-Hodgkin's lymphoma, multiple myeloma or hepatocarcinoma. In a preferred embodiment, the cancer is a prostate or bladder cancer. Patients identified as having altered global histone modification pattern(s) associated with an increased risk of metastasis, recurrence or a therapy resistant cancer can be further selected on that basis for treatment with exogenous or endogenous hormone ablation, optionally supplemented with chemotherapy and/or radiation. In the case of prostate cancer, the hormone ablation is androgen ablation (e.g., treatment with finasteride and other anti-tesosterone or anti-DHT agents).

In another aspect the invention provides methods of assessing the response of a cancer patient to a medical treatment, comprising the steps of contacting a test tissue sample from the individual receiving the treatment with an antibody that specifically binds to a modified histone protein; and determining the global histone modification pattern in the test tissue sample in comparison to a tissue sample taken from the patient before the treatment, or earlier or later in the course of a treatment, or before and after a treatment has been modified. In some embodiments, the therapy is hormonal ablation therapy or chemotherapy or radiation or a pro-apoptosis therapy.

In another aspect, the invention provides a method of providing a prognosis for a cancer by contacting a test tissue sample from an individual at risk for or known to have a cancer with an antibody that specifically binds to a modified histone protein; and determining the global histone modification pattern in the test tissue sample in comparison to a control tissue sample; thereby providing a prognosis for said cancer by identification of an altered global histone modification pattern. In some embodiments, the tissue sample is a tumor biopsy sample. In some embodiments, the cancer or tumor is prostate, bladder, kidney, colon or breast cancer. In preferred embodiments, the individual has less advanced disease (i.e. low grade or stage), the category in which prognostic markers are acutely needed.

In another aspect, the invention provides kits comprising at least two antibodies which each bind a different histone protein modification. In some embodiments, the antibodies are selected from the group consisting of H3 K9 acetylation, H3 K18 acetylation, H4 K12 acetylation, H3 K4 dimethylation, H3 K9diMe, and H4 R3 dimethylation. In some embodiments, the antibodies are labeled with a detectable moiety. In some embodiments, the kits provide reagents for detecting the antibody when bound to a histone protein having the histone protein modification recognized by the antibody. In other embodiments, the kits have instructions relating altered histone modification patterns to an increased or decreased risk of cancer metastasis or progression. In other embodiments, the kits further comprise reagents for use in immunohistochemical methods using the antibodies.

In further embodiments of any of the above aspects, the global histone protein modification is selected from one or more of the group consisting of H3 K9 acetylation, H3 K18 acetylation, H4 K12 acetylation, H3 K4 dimethylation, H3 K9dimethylation, and H4 R3 dimethylation. In still further embodiments of such, the cancer or tumor is prostate, bladder, kidney, colon or breast cancer. The cancer can be a metastatic cancer.

In some embodiments of any of the above aspects, the global histone modification pattern of one, two, three, four, or at least two or three different histone protein modifications is detected. In further embodiments, the at least two different histone protein modifications are selected from the group consisting of H3 K9 acetylation, H3 K18 acetylation, H4 K12 acetylation, H3 K4 dimethylation, a H3 K9dimethylation, and H4 R3 dimethylation. In some preferred embodiments, the histone protein modifications are H3 K4 dimethylation and H3K18 acetylation. In other embodiments, the histone proteins are selected from methylations and acetylations of either or both H3 and H4 histone proteins. In other embodiments, the histone proteins are selected from methylations and acetylations of either or both H2A and H2B histone proteins. The histone proteins and individual are preferably human.

In some embodiments, the altered global histone modification pattern in the individual who has cancer or is suspected of having a cancer is determined by (a) obtaining a tissue sample from a portion the subject wherein the portion has or is suspected of having cancer cells therein; and (b) detecting one, two, three, four or more global histone modifications in the sample to provide a global histone modification pattern and (c) comparing the histone modification pattern to a control or normal global histone modification pattern for a subject to identify an altered global histone modification pattern. In further such embodiments, the global histone modifications are detected using antibodies which specifically bind the histone protein modification of interest. The antibody may be a monoclonal antibody or a polyclonal antibody directed toward the histone modification pattern of interest. In some embodiments, the method further comprises the step of fixing the cells and detecting the global histone modifications in the fixed cells.

In further such embodiments, and in any of the above aspects generally, the immunohistochemical staining uses antibodies to specifically bind the histone protein modification of interest. The antibody may be a monoclonal antibody or a polyclonal antibody directed toward the histone modification pattern of interest. The antibody may be labeled with a detectable label (e.g., a radioactive label, and enzymatic label, a fluorescent label, or chemiluminescent label, or a molecular tag). The label bound to the histone modification of interest may be detected by autoradiography, fluorimetry, luminometry, or phosphoimge analysis. In a preferred embodiment, the global histone modifications are detected for a plurality of individual fixed cells in the sample and the intensity and/or frequency of immunohistochemical staining is determined for each of the plurality such that a frequency distribution of cells according to staining intensities are obtained over an area of interest. Preferably, the area of interest focuses on cells having an altered phenotype suggestive of a cancer. The area may be defined empirically according to the region of the sample having the most intense staining (if a modification positively correlates with the risk, grade, or progression of cancer) or the least intense staining if the modification negatively correlates with the risk, grade, or progression of cancer. The area may be of a predetermined size sufficient to provide a valid measure of staining patterns in the area of interest. Multiple areas may be sampled and compared from each of the tissue samples.

In some embodiments of any of the above aspects, the histone protein modification is selected from one or more of the group consisting of H3 K9 acetylation, H3 K18 acetylation, H4 K12 acetylation, H3 K4 dimethylation, H3 K9dimethylation, and H4 R3 dimethylation. In still further embodiments of such, the cancer or tumor is prostate, bladder, kidney, colon or breast cancer. The cancer can be a metastatic cancer.

In some embodiments of any of the above aspects, the global histone modification pattern of at least two or three different histone protein modifications is detected. In further embodiments, the at least two different histone protein modifications are selected from the group consisting of H3 K9 acetylation, H3 K18 acetylation, H4 K12 acetylation, H3 K4 dimethylation, H3 K9dimethylation, and H4 R3 dimethylation. In some preferred embodiments, the histone protein modifications are H3 K4 dimethylation and H3K18 acetylation. In other embodiments, the histone proteins are selected from methylations and acetylations of either or both H3 and H4 histone proteins. The histone proteins are preferably human. In some embodiments, the selected modifications are modifications of H3 or H4. In some further embodiments, the selected modifications are methylations and/or acetylations of H3 or H4. In other embodiments, the selected modifications comprise a phosphorylation or ubiquinylation of H3 or H4. In further embodiments, the at least two different histone protein modifications are selected from the group consisting of H3 K9 acetylation, H3 K18 acetylation, H4 K12 acetylation, H3 K4 methylation(s), H3 K9 methylation (s), and H4 R3 methylation(s). Characterization of the global histone modification pattern allows the altered global histone modifications which are of diagnostic and prognostic value to be determined.

In some embodiments, the histone modifications used for the analyses are selected according to the predictive power of their altered histone modification patterns with respect to the severity, grade, or likelihood of progression of a cancer. In some embodiments, the histone modification to be analyzed is one whose altered histone modification patterns by themselves, or in combination with a second, third or fourth histone modification pattern, provide a relative risk for an increased likelihood of a more severe outcome or grade of cancer or of metastasis or non-responsiveness to thymidylate synthase treatment on the order of at least 1.5, 2, 3, 4, or 5-fold or more or on the order of from 1.5 to 3-fold, or 1.5 to 4-fold, or 2 to 5-fold.

In other embodiments of any of the above aspects and embodiments, the methods use antibodies which specifically bind to the histone protein modification of interest to detect the modifications. The antibody may be a monoclonal antibody or a polyclonal antibody directed toward the histone modification pattern of interest. In some embodiments, a plurality of global histone modification patterns are determined for the sample. The histones to be analyzed for particular modifications may be first isolated from the sample and detected using immunochemical methods in a fluid medium.

In some embodiments, the ratio of a modified histone protein to the total levels of the histone protein provide a predictive measure based upon altered global histone modification patterns. For instance, a sample may be analyzed using an antibody which detects modified and unmodified forms of a histone protein and an antibody which selectively detects histones have the modification of interest. The ratio of the two in a population of cells or in a sample is determined and is compared to the ratio for a normal cell to establish a predictive ratio of altered global histone protein modification which can be used in the methods according to the invention.

In some embodiments, the altered global histone modification patterns are predictive of whether a cancer or tumor will be refractory to treatment or therapy resistant.

In some embodiments of any of the above, a histone rule is applied wherein cancer patients having a K4 diMe staining value at or above about the 60 percentile and patients have a better prognosis than patients who are below these levels. In some other embodiments, cancer patients having a K18 Ac and K4 diMe staining value which are each at or above about the 35 percentile have a better prognosis than patients who are below these levels.

In some embodiments of any of the above, the tissue is blood and the altered global histone modification pattern for blood cells is determined. In some embodiments, the samples are from patients who have a leukemia or lymphoma and the altered global histone modification pattern includes patterns from leukemic or lymphoma cells. The detection of the global histone modification patterns can be conducted using immunofuorescence staining of the cells followed by FACS sorting and/or scoring and counting of the cells. These methods can provide a frequency distribution of the global histone modification frequencies for the cells of interest in the sample. In such methods it can also be useful to employ other fluorescent markers identifying the particular cell or its phenotype to facilitate in the sorting and counting of the leukemic or lymphoma cells.

In some embodiments of any of the above, the tissue is disaggregated by enzymatic, grinding, or other means and the global histone modification patterns of individual cells are characterized by immunofluorescence staining using the antibodies described herein followed by FACS sorting and/or scoring and counting of the cells which can provide a frequency distribution of the global histone modification frequencies for the sample. In such methods it can also be useful to employ other fluorescent markers identifying the particular cell or its phenotype to facilitate in the sorting and counting of the particular cells of interest.

In some embodiments, the analysis of the modifications are assessed in accordance with an assigned percent rank value (quantile) based on median percent of cells staining relative to a TMA dataset using the SAS system procedure RANK with TIES=LOW option, which assigns the smallest of the corresponding ranks for the ties data values. Each tumor can then be assigned into a low or high level staining group based on its percent rank, including H3K4me2 (<60 vs. ≧60 percent rank), H3K9me2 (<30 vs. ≧30 percent rank for the RTOG TMA or <25 vs. percent rank for the UCLA Stage I/II TMA) and H3K18ac (<35 vs. ≧35 percent rank)(H3K9ac ≧. 10%) or within a range of from 0.8 to 1.2, 0.9 to 1.1, or 0.95 to 1.05 times those values. It is to be understood that the cell staining percentages can be influenced the staining methodologies used. Accordingly, in some embodiments of the invention, the percentages would be equivalent to those obtained herein if obtained by the same methods.

A method of predicting the response of, or selecting a cancer therapy for, of a patient to a thymidylate synthase inhibitor, comprising the steps of (a)contacting a test tissue sample from an individual at risk for or known to have a cancer with an antibody or immunologically active fragment thereof or aptamer that specifically binds to a modified histone protein; and (b) determining the global H3K4 dimethylation, H3K9dimethylation and/or H3K18 acetylation histone modification pattern. In some embodiments, the tissue sample is a tumor biopsy sample of a pancreatic is prostate, bladder, kidney, ovarian, colon or breast cancer. Each tumor can then be assigned into a low or high level staining group based on its percent rank, including H3K4me2 (about <60 vs. ≧60 percent rank), H3K9me2 (about <30 or 25 vs. ≧30 or ≧25 percent rank) and about H3K18ac (<35 vs. ≧35 percent rank) or within a range of from 0.8 to 1.2, 0.9 to 1.1, or 0.95 to 1.05 times those values. It is to be understood that the cell staining percentages can be influenced the staining methodologies used. Accordingly, in some embodiments of the invention, the percentages would be equivalent to those obtained herein if obtained by the same methods.

### DEFINITIONS

Unless otherwise stated, the following terms used in the specification and claims have the meanings given below. It is noted here that as used in this specification and the appended claims, the singular forms “a,” “an,” and “the” include plural reference unless the context clearly dictates otherwise.

Amino acids may be referred to herein by either their commonly known three letter symbols or by the one-letter symbols recommended by the IUPAC-IUB Biochemical Nomenclature Commission.

“Global histone modification” refers to patterns of histone protein modification that are not confined to promoter regions but that encompass large areas of chromatin, including non-promoter regions. Global histone modification patterns may be determined by any means known in the art, including immunological methods and the like employing antibodies, apatamers, and immunologically active fragments of the antibodies which can bind to the histone modification of interest. Immunohistochemical and immunocytological methods may be used in detecting the modified histones or staining the cells to establish a global histone modification pattern and the percent of cells staining for the modification. Mass spectroscopic and electrochemical means may also be used. All possible methods of measuring global patterns of histone modifictions including non-antibody-based protocols may be used, software programs providing for detection of epigenetic patterns recognizable when staining tissue histone markers bringing to attention certain morphological and phenotypic patterns correlating with the staining patterns of the histone marks can be incorporated into the methods. In some instances, in the case of a single marker like H3k9me2, a 10% >=may be used as a cut off below which the score represents poor prognosis and in a binary fashion could be considered. In general, the cutoff percentages would include their equivalents as determined or detected by another method. In preferred embodiments, the cut-off values are established which demarcate groups differing substantially in their relative survival rates, prognosis, or responsive to therapy. For instance, a cut-off which provides a difference in the relative likelihood of survival, or survival in response to a therapy, of at least 20%, 30%, 40, 50%, 60% can be selected for survival periods of 6 months, 1 year, 2 years, 3 years, 4 years, 5 years, 10 years or longer.

“Histone” refers to DNA binding structural proteins of chromosomes. Histones have a high proportion of positively charged amino acids such as lysine and arginine, which aids in DNA binding. The five main types of histones fall into two groups: nucleosomal histones H2A, H2B, H3, H4; and H1 histones. “Modified histone protein” refers to a histone protein with one or more of the following chemical modifications which include, but are not limited to, lysine acetylation, lysine methylation (mono-, di-, and trimethylation), lysine ubiquitylation, arginine methylation (mono-, di-, symmetric and asymmetric methylation), serine/threonine/tyrosine phosphorylation.

With regard to amino acid sequence, histone H3 includes the proteins of SEQ ID NO:1 and SEQ ID NO:2 (see, Swiss Prot Acc No: Q93081 (which is incorporated by reference in its entirety with respect to the sequence itself) and the naturally occurring variants including, but not limited to, the modified histone proteins thereof, as well as proteins which are substantially identical thereto, and in particular, also lack the N-terminal methionine residue at position 1 of the above sequences (e.g., a post-translational loss of the N-terminal methionine residue). Modified histone proteins are well known in the art. For instance, the suitable histone protein modifications may include, but are not limited to, any one or more listed below. Exemplary protein modifications for possible use according to the invention are set forth below.

Histone Protein H3 with Potential Sites of Modification

Swiss Prot Acc No: Q93081

| Pos. | AA | Mod. |
|---|---|---|
| 2 | R | Me |
| 3 | T | P |
| 4 | K | Me |
| 9 | K | Ac Me |
| 10 | S | P |
| 11 | T | P |
| 14 | K | Ac Me |
| 17 | R | Me |
| 18 | K | Ac |
| 23 | K | Ac Me |
| 26 | R | Me |
| 27 | K | Ac Me |
| 28 | S | P |
| 32 | T | P |
| 36 | K | Me |
| 37 | K | Me |
| 56 | K | Me |
| 79 | K | Me |
| 115 | K | Ac |
| 118 | T | P |
| 122 | K | Ac |
| 128 | R | Me |

### Selected Histone Modifications

| | | | Single | | Double | | Triple | |
|---|---|---|---|---|---|---|---|---|
| | | | Mod | | Mod | | Mod | |
| Histone | Site | Modification | mAb | pAb | mAb | pAb | mAb | pAb |
| H3 | | un-modified | X | | | | | |
| 14-494 | non- | Acetyl | | X | | | | |
| | spec | | | | | | | |
| K4 | | Methyl | | X | X | X | | X |
| K9 | | Methyl | | X | X | X | | |
| K9 | | Acetyl | | X | | | | |
| S10 | | Phos | X | X | | | | |
| K14 | | Acetyl | | X | | | | |
| R17 | | Methyl | | | | X | | |
| K18 | | Acetyl | | X | | | | |
| K23 | | Acetyl | | X | | | | |
| R26 | | Methyl | | | | X | | |
| K27 | | Acetyl | | X | | | | |
| K27 | | Methyl | | | | X | | |
| S28 | | Phos | | X | | | | |
| K36 | | Methyl | | | | X | | |
| K79 | | Methyl | | | | X | | |
| S10/ | | Phos/Acetyl | | X | | | | |
| K14 | | | | | | | | |
| K4/K9 | | dimethyl | | | | X | | |

In the above tables, “Me” refers to methyl modifications, “Ac” to acetyl modifications, “P” or “phos” refer to phosphorylation modifications, and “Ub” to ubiquinylations. Where Me is indicated it may be a mono-, di, or trimethylation. Where two modifications are listed for a particular protein residue, they can be alternative modifications.

The residue position of the tables is with respect to the positions of SEQ ID Nos: 1 to 6, renumbered without the N-terminal methionine (i.e, the residue position of SEQ ID NOs: 1 to 6 minus one). In preferred embodiments, the histone proteins of SEQ ID NOs:1 to 6 to be detected lack an N-terminal methionine residue.

Histone deacetylase inhibitors for use according to the invention include, but are not limited to, vorinostat, FK228, PXD101, PCI-24781, ITF2357, MGCD0103, MS-275, valproic acid and LBH589 (see, Tan et al., Journal of Hematology & Oncology 2010, 3:5). Accoringly, the inhibitor can be an (a) organic hydroxamic acids (e.g., Trichostatin A (TSA) and suberoylanilide bishydroxamine (SAHA)) (b) short-chain fatty acids (e.g., butyrates and valproic acid (VPA)), (c) benzamides (e.g., MS-275), (d) cyclic tetrapeptides (e.g., trapoxin), and (e) sulfonamide anilides. Agents include LBH589 (panobinostat), PCI24781 (CRA-024781), LAQ824 I, II, PXD101 (belinostat), ITF2357, SB939, JNJ-16241199 (R306465), m-carboxycinnamic acid bishydroxamide (CBHA), Scriptaid, Oxamflatin, Pyroxamide, Cyclic hydroxamic acid containing peptides (CHAPs), AN-9, OSU-HDAC42, Benzamides MS-275 (entinostat), MGCD0103, Pimelic diphenylamide, M344, N-acetyldinaline (CI-994), Cyclic tetrapeptides Apicidine, Trapoxins, HC-toxin, Chlamydocin, Depsipeptide (FR901228 or FK228) (romidepsin), sulfonamide anilides, N-2-aminophenyl-3-[4-(4-methylbenzenesulfonylamino)-phenyl1-2-propenamide, Depudecin, NDH-51 and KD5150.

“Immunohistochemistry” refers to the use of antibodies or aptamers to detect proteins in biological samples such as cells and tissue sections. The detection methods of the present invention can be carried out, for example, using standard immunohistochemical techniques known in the art (reviewed in Gosling, Immunoassays: A Practical Approach, 2000, Oxford University Press). Detection is accomplished by labeling a primary antibody or a secondary antibody with, for example, a radioactive isotope, a fluorescent label, an enzyme or any other detectable label known in the art. Visual grading of tissue sections by intensity of staining is well known in the art. Standard controls from tumor and healthy tissue samples are routinely used by those of skill in the art to control for variation among samples and reagents. Moreover, negative controls that do not include primary antibodies specific for the desired target (i.e., histone) are used routinely as controls. Van Diest et al., Anal. Quant. Cytol. Histol. 18(5):351-4 (1996), discloses that even inexperienced observers can, with a few minutes' training, reproducibly grade breast tumor sections on a 0-4 scale based on immunohistochemical staining intensity. Thus, those of skill in the art can grade samples based on a scale (e.g., 0-4 or other), based on percent staining, or based on a simple determination of positive or negative. Methods of immunohistochemical staining are exemplified in the specification. In some embodiments, the frequencies of tissue samples in which an indicated percent or degree of cell staining occurs are ascertained for each modification. Those of ordinary skill in the art appreciate the use of standard controls from tumor and healthy tissue samples to control for variation among samples and reagents. Moreover, negative controls that do not include primary antibodies specific for the desired target can be used routinely to control for background at the time the application was filed. Methods of immunohistochemical scoring are also well known in the art. In some embodiments, immunohistochemical scoring is on a scale from 0 to 4 or 1 to 4. For example, Van Diest et al., Anal. Quant. Cytol. Histol. 18(5):351-4 (1996) disclose that even inexperienced observers can, with a few minutes' training, reproducibly grade breast tumor sections on a 0-4 scale based on immunohistochemical staining intensity.” A person of ordinary skill would know how to adapt the method to use aptamers in place of the antibodies.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide. Labels may be conjugated directly to the biorecognition molecules, or to probes that bind these molecules, using conventional methods that are well known in the arts. Multiple labeling schemes are known in the art and permit a plurality of binding assays to be performed simultaneously. Different labels may be radioactive, enzymatic, chemiluminescent, fluorescent, quantum dot, or others. Methods of covalently or noncovalently conjugating labels to antibodies are well known to one of ordinary skill in the art. Methods of detecting proteins and modified proteins by use of labeled antibodies are also well known to persons of ordinary skill in the art.

“Cancer” refers to human cancers and carcinomas, sarcomas, adenocarcinomas, lymphomas, leukemias, etc., including but not limited to solid tumors and lymphoid cancers, kidney, breast, lung, kidney, bladder, colon, ovarian, prostate, pancreas, stomach, brain, head and neck, skin, uterine, testicular, esophagus, and liver cancer, lymphoma, including but not limited to non-Hodgkins and Hodgkins lymphoma, leukemia, and multiple myeloma. In preferred embodiments, the cancer is an adenocarcinoma, a pancreatic cancer, a breast cancer, a prostate cancer, a lung cancer, or a kidney cancer. Specific types of cancers including malignant tumors, either primary or secondary, for which prognosis and 5-FU responsiveness can be assessed according to the invention include, but are not limited to, bone cancer, cancer of the larynx, gall bladder, rectum, head and neck, bronchi, basal cell carcinoma, squamous cell carcinoma of both ulcerating and papillary type, metastatic skin carcinoma, osteosarcoma, Ewin's sarcoma, reticulum cell sarcoma, myeloma, giant cell tumor, small-cell lung tumor, islet cell tumor, primary brain tumor, acute and chronic lymphocytic and granulocytic tumors, hair-cell tumor, adenoma, hyperplasia, medullary carcinoma, pheochromocytoma, mucosal neuromas, cervical cancer, neuroblastoma, retinoblastoma, soft tissue sarcoma, malignant carcinoid, rhabdomyosarcoma, Kaposi's sarcoma, oteogenic and other sarcoma, renal cell tumor, gliobastoma multiforma, malignant melanomas, epidermoid carcinomas.

5-FU therapy includes, but is not limited to, treatment with 5-FU and prodrugs of 5-FU and combination therapies with 5-FU and its prodrugs (e.g., with Leucovorin).

“Biological sample” includes sections of tissues such as biopsy and autopsy samples, and frozen sections taken for histologic purposes. Tissue, cultured cells, e.g., primary cultures, explants, and transformed cells. In one embodiment, the biological sample is a tissue sample prepared for immunohistochemistry. In another embodiment, the biological sample is a tissue sample prepared as a tissue microarray (TMA) for high throughput screening. A biological sample is typically obtained from a eukaryotic organism, most preferably a human or a mammal such as a primate e.g., chimpanzee; cow; dog; cat; a rodent, e.g., guinea pig, rat, Mouse; rabbit; or a bird; reptile; or fish.

A “biopsy” refers to the process of removing a tissue sample for diagnostic or prognostic evaluation, and to the tissue specimen itself. Any biopsy technique known in the art can be applied to the diagnostic and prognostic methods of the present invention. The biopsy technique applied will depend on the tissue type to be evaluated, the size and type of the tumor, among other factors. Representative biopsy techniques include excisional biopsy, incisional biopsy, needle biopsy, surgical biopsy, and bone marrow biopsy. An “excisional biopsy” refers to the removal of an entire tumor mass with a small margin of normal tissue surrounding it. An “incisional biopsy” refers to the removal of a wedge of tissue that includes a cross-sectional diameter of the tumor. A diagnosis or prognosis made by endoscopy or fluoroscopy can require a “core-needle biopsy” of the tumor mass, or a “fine-needle aspiration biopsy” which generally obtains a suspension of cells from within the tumor mass. Biopsy techniques are discussed, for example, in Harrison's Principles of Internal Medicine, Kasper, et al., eds., 16th ed., 2005, Chapter 70, and throughout Part V.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The teens variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990))

For preparation of antibodies, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (U.S. Pat. No. 4,946,778, U.S. Pat. No. 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

Treatments to be used in the case where the patient is resistant to 5-FU or a thymidylate synthase inhibitor or has a poorer expected survival based upon their global histone modification pattern include drugs impacting dhfr pathway, hormone therapy, immunotherapy, RNAi therapeutics, radiation therapy, nutraceutical therapies, “meditation” Therapy, any therapy in general where cellular energy metabolism is impacted and or drugs contributing to shifting global patterns of histone modifications from low levels of modification to higher level of modifications and I think this could be an all encompassing way of describing response predictions to any drug impacting the shift from low-to-high levels of cellular histone modifications.

Subjects whose histone modification patterns indicates they are unlikely to be responsive to 5-FU or another thymidylate synthase inhibitor can be treated with an additional or alternative therapy to 5-FU or the thymidylate synthase inhibitor which would include, but not be limited to, treatment with another chemotherapeutic agent, an immunotherapy, a radiation therapy, an antisense therapy, RNAi therapy, a hormone therapy, drugs impacting the dhfr pathway, and an anti-metabolate therapy (e.g., folate inhibitor, methotrexate), a taxane (e.g., paclitaxel, a taxol), Abraxanes, kinase inhibitors, particularly inhibitors of c-met, MEK, Apo2L/TRAIL, EGFR (inhibitors of both internal and external domains of EGFR), anti-VEGF therapies, and anti-IGF1R & IGF2R therapies. In addition, Nutraceutical therapies or any therapy in general where cellular energy metabolism is impacted and/or drugs contributing to shifting global patterns of histone modifications from low levels of modification to higher level of modifications are administered. Subjects whose global histone modifications indicate they will have a worse prognosis can also be administered more aggressive treatments, including any one or combinations of the above therapies.

In one embodiment, the antibody is conjugated to an “effector” moiety. The effector moiety can be any number of molecules, including, but not limited to, labeling moieties such as radioactive labels or fluorescent labels, or can be a therapeutic moiety. In one aspect the antibody modulates the activity of the protein.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only those polyclonal antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g.,

Harlow & Lane, Using Antibodies, A Laboratory Manual (1998) for a description of immunoassay formats and conditions that can be used to determine specific immunoreactivity).

The teams “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, λ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

### EXAMPLES

The following examples are offered to illustrate, but not to limit the claimed invention.

### Example 1

### The Prognostic and Predictive Value of Three Histone Modifications in Pancreatic Adenocarcinoma

METHODS: Tissue microarrays (TMAs) from two large pancreatic adenocarcinoma cohorts were examined, including a 195 patient cohort from RTOG 9704, a multi-center phase III randomized treatment trial comparing adjuvant gemcitabine versus 5-fluorouracil (5-FU), and a 140 patient cohort of Stage I or II cancer from UCLA Medical Center. Immunohistochemistry for three histone modifications (H3K4me2, H3K9me2 and H3K18ac) was performed. Positive tumor cell staining of the histone modifications was used to classify patients into low and high staining groups, which were related to clinicopathologic parameters and clinical outcome measures.

RESULTS: Low cellular levels of H3K4me2, H3K9me2 or H3K18ac were each significant and independent predictors of poor survival in univariate and multivariate models, with combined low levels of H3K4me2 and/or H3K18ac predicting the worst overall survival (hazard ratio [HR], 2.54; 95% confidence interval [CI], 1.53-4.22; P=0.0003) in the UCLA Stage I/II TMA. In subgroup analyses, histone levels were predictive of survival for only those patients with node-negative cancer or for those patients receiving adjuvant 5-FU, but not gemcitabine, in RTOG 9704.

Pancreatic adenocarcinoma patients and tissue microarrays. The RTOG 9704 pancreatic cancer tissue microarray (TMA) consisted of 229 cases of pancreatic adenocarcinoma obtained from patients enrolled in RTOG 9704, a phase III randomized post-operative adjuvant treatment trial comparing 5-fluorouracil (5-FU) to gemcitabine before and after chemoradiation (Regine et al., Jama 299:1019-26 (2008)). In RTOG 9704, all patients received adjuvant chemotherapy (5-FU or gemcitabine) for durations of one month before and three months following chemoradiation therapy, which included 5-FU infusion as a radiation sensitizer. Clinicopathologic factors were collected as part of patient enrollment, as were treatment schedules and follow-up clinical information including toxicity, overall survival and disease-free survival. The UCLA Stage I/II pancreatic cancer TMA consisted of 140 cases of AJCC stage I or II pancreatic adenocarcinoma from the UCLA Department of Pathology and Laboratory Medicine archives, representing patients who underwent complete gross resection of tumor at UCLA Medical Center between 1987 and 2005. All work was performed with appropriate institutional review board approvals.

Immunohistochemistry. A standard 2-step indirect IHC staining method was used for all antibodies as previously described (Seligson et al., Am J Pathol 174:1619-1628 (2009)) using the DAKO Envision System (Carpenteria, Calif.). Primary rabbit anti-histone polyclonal antibodies were applied for 60 min at room temperature including H3K9me2 (Upstate/Millipore, Billerica, Mass.) at 1:800, H3K18ac (Upstate) at 1:200 and H3K4me2 (Abcam, Cambridge, Mass.) at 1:800. Control staining was performed in identical fashion without primary antibody. Semi-quantitative assessment of the percentage of tumor cells with positive nuclear staining (range 0-100%) was independently performed by two of three pathologists (D. D., N. D. or A. M.), who were blinded to all clinicopathologic and outcome variables. For each patient tumor, three representative 0.6 mm cores (RTOG 9704 TMA) or two representative 1.0 mm diameter cores (UCLA Stage I/II TMA) were scored and used to calculate the median percent of cells staining, and included all scores from both pathologists.

Statistical Analysis. Specific histone cut-offs were previously shown to predict survival in subsets of patients with multiple types of carcinoma, including prostate, lung and kidney (Seligson et al., Am J Pathol 174:1619-1628 (2009); Seligson et al., Nature 435:1262-6 (2005)). In order to standardize and apply these same cut-offs in both pancreatic cancer TMAs, each tumor was assigned a percent rank value (quantile) based on median percent of cells staining relative to its TMA dataset using the SAS system procedure RANK with TIES=LOW option, which assigns the smallest of the corresponding ranks for the ties data values. Each tumor was then assigned into a low or high level staining group based on its percent rank, including H3K4me2 (<60 vs. ≧60 percent rank), H3K9me2 (<30 vs. ≧30 percent rank for the RTOG TMA or <25 vs. ≧25 percent rank for the UCLA Stage I/II TMA) and H3K18ac (<35 vs. ≧35 percent rank). Survival estimates were generated and visualized using the Kaplan-Meier method and survival curves were compared using the log-rank test. Multivariate Cox proportional hazards models were used to test statistical independence and significance of multiple predictors. Overall survival time was measured from the date of randomization (RTOG 9704 TMA) or date of surgery (UCLA Stage I/II TMA) to the date of death due to any cause or last follow-up. Disease-free survival time was only determined for RTOG 9704 and was measured from the date of randomization to the date of first disease-free failure event defined as local or regional disease relapse, distant disease, second primary or death due to any cause.

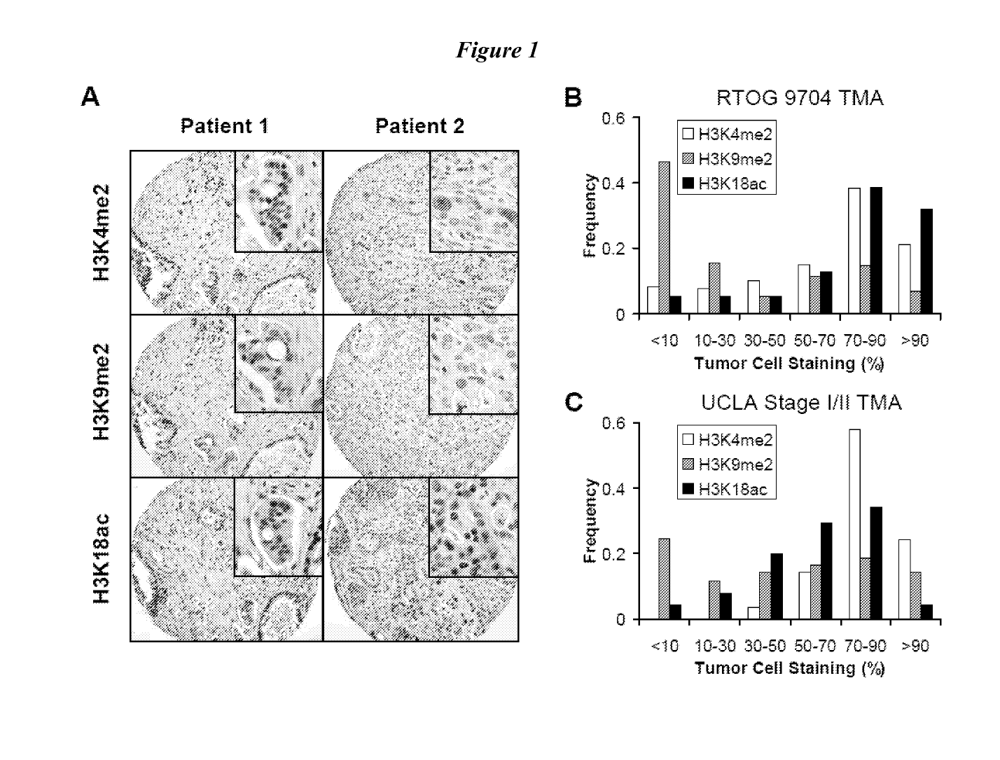

Cellular histone modification levels in pancreatic adenocarcinoma TMAs. Cellular levels of H3K4me2, H3K9me2 and H3K18ac were examined in two different pancreatic adenocarcinoma TMAs by immunohistochemistry using antibodies specific to the modified histone residues. The first TMA examined consisted of patients enrolled in RTOG 9704, a phase III multi-center, randomized controlled trial comparing gemcitabine versus fluorouracil adjuvant chemotherapy in conjunction with fluorouracil chemoradiation following complete gross resection of pancreatic adenocarcinoma (Regine et al., Jama 299:1019-26 (2008)). From an original 229 treatment-naive resected tumors in the TMA, 195 had diagnostic tumor present for immunohistochemical evaluation, including 103 patients in the fluorouracil treatment arm and 91 (or 92 for H3K9me2) in the gemcitabine treatment arm. The second TMA consisted of 140 patients with AJCC Stage I or II pancreatic adenocarcinoma who underwent complete gross surgical resection at UCLA Medical Center. Representative staining for each of the three histone modifications is shown in FIG. 1. Absence of nuclear staining indicates a bulk decrease in a given cell, and thus assesses the cellular heterogeneity of that histone modification. Tumors ranged from 0 to 100% percent cell staining for each of the three histone modifications, with H3K4me2 and H3K18ac skewed towards overall higher percent cell staining and H3K9me2 skewed towards overall lower percent cell staining (FIG. 1).

Prior work in prostate, lung and kidney cancers identified and validated the “histone rule,” a classifier that divides patients into high and low risk groups based on the percent rank staining of each histone modification (Seligson et al., Am J Pathol 174:1619-1628 (2009); Seligson et al., Nature 435:1262-6 (2005)) (which herein are specifically incorporated by reference with respect to their disclosure of such rule). For each of our two pancreatic cancer TMAs, we used this same histone rule to classify patients into low or high staining groups. Combinations of two or more histone modifications were also used to classify patient groups. No statistically significant associations were found between baseline clinicopathologic parameters and histone grouping status in the RTOG 9704 TMA, although low H3K4me2 and node-negative status (N0) approached statistical significance (p=0.051, Chi-square test; data not shown). Likewise, no significant associations were found between baseline parameters and histone groups in the UCLA Stage I/II TMA, with the exception of a highly significant association between low H3K4me2 and low pathologic T-stage (p=0.007, Chi-square test; data not shown). While low H3K4me2 was associated with worse prognosis in both TMAs (see below), N0 status (RTOG 9704 TMA) or low pathologic T-stage (UCLA Stage I/II TMA) was paradoxically associated with better prognosis. These data indicate that patient histone groups are decoupled from established pathologic staging parameters that predict clinical outcome.

Histone modification levels predict survival in pancreatic cancer. In the RTOG 9704 TMA, low H3K4me2 (<60 percent rank) or low H3K9me2 (<30 percent rank) were significant and independent predictors of worse overall and disease-free survival by multivariate proportional hazards analyses, while low H3K18ac (<35 percent rank) was a significant predictor of worse disease-free and trended toward worse overall survival (Table 1). Kaplan-Meier survival curves visualized significant associations between low levels of H3K4me2 or H3K9me2 and worse overall survival (data not shown). Combinations of two or more histone modifications could also be used to group patients where combined low level histone groups were again significant and independent predictors of worse overall and disease-free survival (Table 1). These data indicate that cellular histone modification levels are strong prognostic markers in the RTOG 9704 pancreatic cancer cohort.

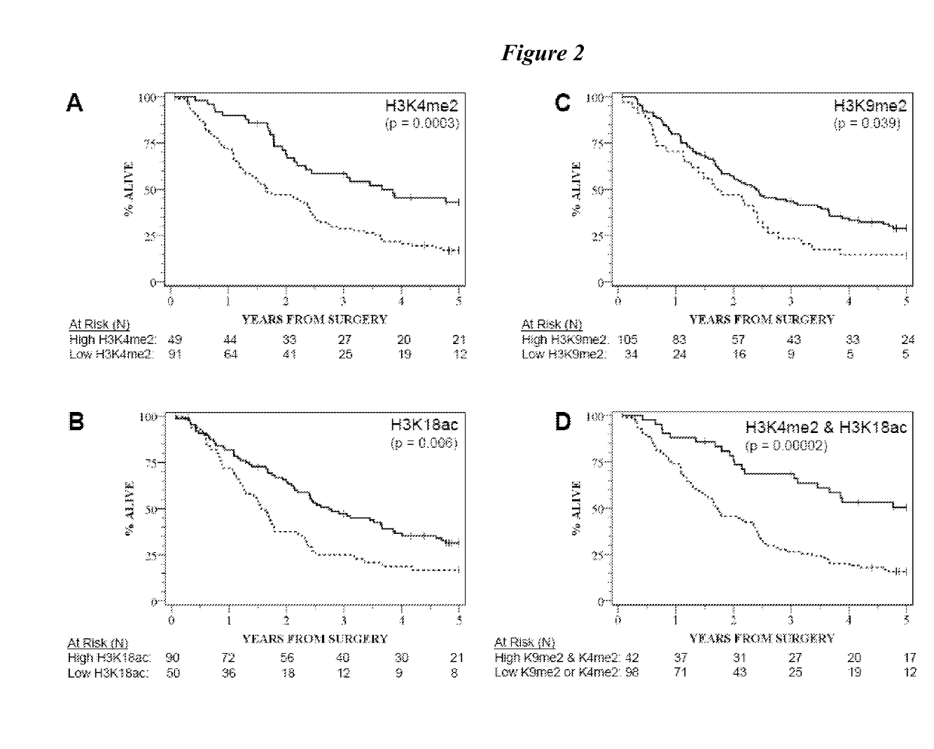

To independently validate the results of the RTOG 9704 TMA, histone groups were separately examined in the UCLA Stage FIT pancreatic cancer TMA. Low level groups for H3K4me2 and H3K18ac were significant and independent predictors of worse overall survival in the UCLA Stage I/II TMA by multivariate Cox regression analyses, while low level H3K9me2 trended toward worse overall survival (Table 2). Kaplan-Meier survival curves confirmed significantly reduced median survival times for each low histone grouping (FIG. 2), including low versus high H3K4me2 (1.68 years, 95% CI 1.02-2.33 versus 3.66 years, 95% CI 1.84-5.49; P =0.0003), low versus high H3K9me2 (1.68 years, 95% CI 0.74-2.61 versus 2.39 years, 95% CI 1.76-3.03; P=0.039) and low versus high H3K18ac (1.56 years, 95% CI 1.25-1.86 versus 2.74 years, 95% CI 2.04-3.43; P=0.006). Combined low H3K4me2 and/or low H3K18ac versus high H3K4me2 and high H3K18ac was the most highly significant and independent predictor of survival by multivariate proportional hazards analysis (Table 2), with respective median survival times of 1.70 years (95% CI 1.20-2.20) versus >5 years (confidence interval cannot be determined), as determined by Kaplan-Meier survival analysis (log rank test, p=0.00002, FIG. 2). Therefore, the UCLA Stage I/II pancreatic cancer cohort validates findings from the RTOG cohort, indicating that cellular histone modifications are significant and independent prognostic markers for grossly resected pancreatic adenocarcinoma.

Histone modifications predict prognosis in node-negative pancreatic cancer. Tumor stage, lymph node involvement or histologic grade are important predictors of clinical outcome in pancreatic cancer (Garcea et al., Jop 9:99-132 (2008)). However, even within these useful clinicopathologic groups there remains a wide range of survival outcomes. To determine whether histone groups might further classify patients into distinct prognostic groups, we performed subgroup analysis of histone levels after first stratifying patients based on T-stage, N-stage or histologic grade. Histone groups were significant and independent predictors of worse overall survival for patients with node-negative pancreatic cancer in the UCLA Stage I/II TMA, and most significantly (HR=5.00, 95% CI 2.25−11.1; p=0.00007) for the patient group defined by low levels of H3K4me2 and/or H3K18ac. By contrast, histone groups did not discriminate differences in survival for the subset of patients with node-positive pancreatic cancer (data not shown). Strikingly, this result was also validated in the RTOG 9704 TMA where H3K4me2, H3K18ac or combinations of both were again significant predictors of overall survival in node-negative, but not node-positive, pancreatic cancer as determined by multivariate Cox regression analyses (data not shown). These findings indicate cellular histone levels may be best utilized as prognostic markers for node-negative pancreatic cancer.

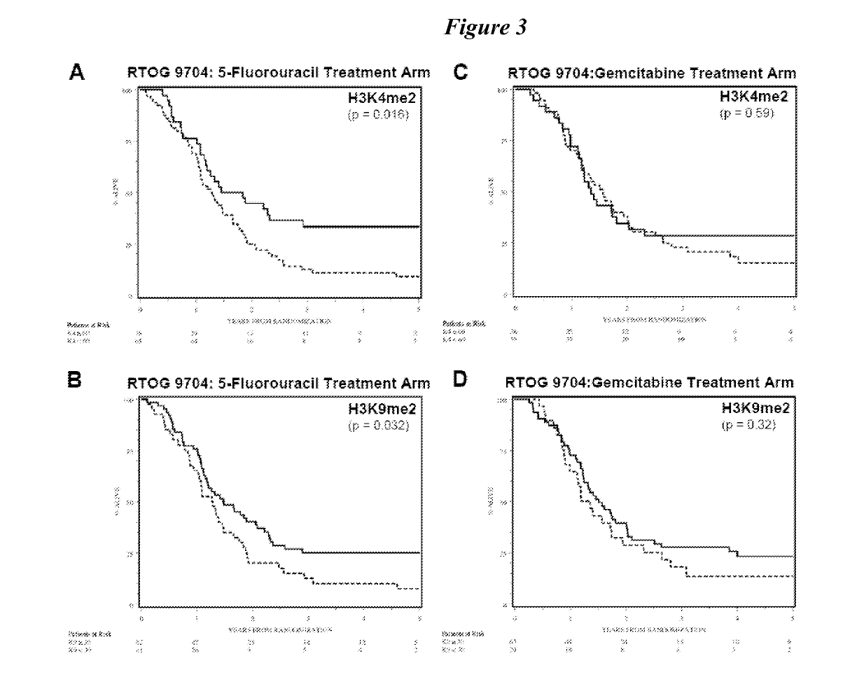

Histone modification levels predict response to adjuvant 5-FU chemotherapy. We next examined whether histone levels were able to predict response to 5-FU or gemcitabine adjuvant chemotherapy in the RTOG 9704 TMA. First, we stratified patients based on their histone groups and performed Kaplan-Meier survival analysis to compare adjuvant treatments. For each of the high level histone subgroups there were no significant differences in overall or disease-free survival for patients receiving either gemcitabine or 5-FU adjuvant chemotherapy (data not shown). In contrast, for the low H3K4me2 subgroup or low H3K18ac subgroup there was worse disease-free survival for patients receiving 5-FU versus gemcitabine (log rank tests, p=0.014 and p=0.015, respectively), as well as a non-significant trend towards worse overall survival for 5-FU versus gemcitabine in the low H3K4me2 subgroup (data not shown). Next, we stratified patients based on adjuvant therapy and performed Kaplan-Meier survival analyses to compare each of the low versus high histone groups. Low levels of H3K4me2 or H3K9me2 were significantly associated with worse overall survival in the subgroup of patients receiving 5-FU, but not in the subgroup of patients receiving gemcitabine (FIG. 3). Univariate hazards models also indicated low levels of H3K4me2, H3K9me2 or H3K18ac were associated with worse overall and disease-free survival in the subgroup of patients receiving 5-FU, but not the subgroup receiving gemcitabine (data not shown). These results indicate that low histone levels identify those pancreatic cancer patients less likely to derive survival benefit from 5-FU adjuvant therapy.

DISCUSSION. We have analyzed multiple histone modifications in two large pancreatic adenocarcinoma patient cohorts and found that cellular patterns of histone modifications provide additional prognostic and predictive information beyond established clinicopathologic criteria. Similar to already published results for H3K27me316, we found a significant association between reduced cellular levels of H3K4me2, H3K9me2 or H3K18ac and worse prognosis in pancreatic adenocarcinoma. Our work here and studies in other cancers (Seligson et al., Am J Pathol 174:1619-1628 (2009); Seligson et al., Nature 435:1262-6 (2005)) highlight the widespread applicability of cellular histone modification levels as prognostic markers, and suggest that a standardized immunohistochemical procedure for detecting cellular histone levels can provide additional non-redundant prognostic information.

Survival varies widely for patients with pancreatic adenocarcinoma, even within subsets of patients stratified by clinicopathologic criteria such as tumor grade, stage or lymph node status. In both of our TMA datasets, histone levels were prognostic for the subset of patients with node-negative pancreatic cancer. This is consistent with previous reports where the prognostic value of histone modifications were largely confined to less aggressive or early-stage cancers, including lower Gleason score prostate cancer13, lower stage lung cancer (Seligson et al., Am J Pathol 174:1619-1628 (2009)) and localized kidney cancer (Seligson et al., Am J Pathol 174:1619-1628 (2009)). Thus, cellular histone levels are best suited as biomarkers when used in conjunction with routine clinicopathologic grading and staging information.

Genome-wide profiling studies comparing normal versus cancer cells indicate a dynamic interplay between active or repressive histone markers and altered gene expression (Ke et al., PLoS ONE 4:e4687 (2009)). While changes in a histone modification at a particular genetic locus may predictably alter gene expression, the consequences of global changes in the levels of multiple histone modifications is more difficult to forecast given their potential opposing functional effects on transcriptional activity and our incomplete understanding of their distribution across the cancer genome. While decreased levels of nearly all histone modifications studied thus far have been linked to worse prognosis, these same histone modifications are variably associated with transcriptional activation (e.g., H3K4me2 and H3K18ac) or transcriptional silencing (H3K27me3, H3K9me2) (Esteller, M., Nat Rev Genet 8:286-98 (2007)). One possible explanation is that, similar to global DNA hypomethylation in cancer (Eden et al., Science 300:455 (2003)), bulk reductions in histone modifications may lead to genomic instability. In support of this hypothesis, prostate cancer cell lines with large differences in H3K9me2 levels have been shown to alter the distribution H3K9me2 almost exclusively at repetitive DNA elements and not gene promoters (Seligson et al., Am J Pathol 174:1619-1628 (2009)). Likewise, global losses of H4K16ac and H4K20me3 in cancer cells have been shown to occur primarily at repetitive elements in combination with DNA hypomethylation (Fraga, et al.: Nat Genet 37:391-400 (2005)). Experimentally, reduction in H3K9me2 levels by knockdown of the histone methyltransferase G9a has been shown to induce chromosomal instability, while having little impact on gene expression in cancer cell lines (Kondo et al., PLoS ONE 3:e2037 (2008)). Further studies are needed to determine the global distribution of histone modifications and the underlying reasons for their reduced levels in subsets of more clinically aggressive pancreatic cancer, as well as studies that establish the direct effects of altered histone modification levels on genomic instability and gene transcription.

The present approach for adjuvant chemotherapy in resected pancreatic cancer primarily involves the choice between gemcitabine versus 5-FU, with an evolving consensus that gemcitabine provides improved survival benefit (Ueno H. K., T., J Hepatobiliary Pancreat Surg 15:468-472 (2008)). Of note, however, RTOG 9704 concluded that adjuvant gemcitabine provided a non-statistically significant survival benefit over adjuvant 5-FU in the setting of fluorouracil-based chemoradiation (Regine et al., Jama 299:1019-26 (2008)), a finding that highlights the need for predictive biomarkers better able to inform treatment decisions. Towards this end, accumulating data suggest the levels of one or more mediators of drug transport or metabolism may be useful in predicting response to gemcitabine 24-27 .or 5-FU28 chemotherapy in cancer. Our data here indicate cellular histone modification levels are a novel class of biomarkers for predicting response to 5-FU. In keeping with our observation that lower H3K18ac levels are associated with worse response to 5-FU, certain histone deacetylase inhibitors (which will act to increase global levels of H3K18ac) have been shown to act in synergy with 5-FU to increase its cytotoxic and growth inhibitory effects in cancer cell lines (Lee et al., Mol Cancer Ther 5:3085-95 (2006); Tumber et al., Cancer Chemother Pharmacol 60:275-83 (2007)). This appears to be at least due in part to reduction in the levels of thymidylate synthase (Lee et al., Mol Cancer Ther 5:3085-95 (2006); Fazzone et al., Int J Cancer Epub (PMID: 19384949) (2009)), which has been associated with resistance to 5-FU chemotherapy. By extension, cellular levels of H3K18ac may be useful in identifying patients more likely to benefit from the addition of an HDAC inhibitor to 5-FU chemotherapy.

Here we have demonstrated that low cellular histone modification levels identify pancreatic cancer patients less likely to derive survival benefit from adjuvant 5-FU chemotherapy, while high cellular histone levels identify patients who derive similar survival benefit from the use of either adjuvant gemcitabine or 5-FU chemotherapy. We conclude that cellular histone modification levels represent a novel category of biomarkers able to predict response to adjuvant 5-fluorouracil chemotherapy in resected pancreatic cancer, and with potential applicability to the neoadjuvant setting or advanced pancreatic cancer. More generally, cellular histone modification levels may also prove to be useful predictive biomarkers in other malignancies (i.e., colorectal or breast cancer) where 5-fluorouracil is utilized as a standard chemotherapy.

### Example 2

### Global Levels of Histone Modifications Predict Prognosis in Different Cancers

This example discloses subject matter found in the priority application, U.S. Provisional Application Ser. No. 61/169,212, filed on Apr. 14, 2009, and also now in Seligson et al., The American Journal of Pathology, Vol. 174, No. 5:1619-28, May 2009, the contents of each of are incorporated by reference herein in their entirety.)

Cancer cells exhibit alterations in histone modification patterns at individual genes and globally at the level of single nuclei in individual cells. We demonstrated previously that lower global/cellular levels of histone H3 lysine 4 dimethylation (H3K4me2) and H3K18 acetylation (ac) predict higher risk of prostate cancer recurrence. Here we show that the cellular levels of H3K4me2 and H3K18ac also predict clinical outcome in lung and kidney cancer patients, with lower levels predicting significantly poorer survival probabilities in both cancers. We also show that lower cellular levels of H3K9me2, a modification associated with both gene activity and repression, is also prognostic of poorer outcome in prostate and kidney cancers. The predictive power of the histone modifications was independent of tissue-specific clinico-pathological variables, proliferation marker Ki67 or p53 tumor suppressor mutation. Chromatin immunoprecipitation experiments indicated that the lower cellular levels of histone modifications in more aggressive cancer cell lines correlate with lower levels of the modifications at DNA repetitive elements but not with gene promoters genomewide. Our results suggest that lower global levels of histone modifications are predictive of a more aggressive cancer phenotype, revealing a surprising commonality in prognostic epigenetic patterns of adenocarcinomas of different tissue origins.

Cancer is a disease of genetic and epigenetic alterations. Epigenetics include the interrelated processes of DNA methylation and histone modifications, aberrations of which occur commonly in human cancer (Baylin, S. B., Ohm, J. E., Nat Rev Cancer 6:107-116 (2006); Feinberg, A. P., Tycko, B., Nat Rev Cancer, 4:143-153 (2004); Jones, P. A., Baylin, S. B., Cell 128:683-692 (2007)). In the case of histone modifications, these aberrations may occur locally at gene promoters by inappropriate targeting of histone modifying enzymes, leading to improper expression or repression of individual genes that play important roles in tumorigenesis. For instance, the E2F transcription factor recruits the tumor suppressor retinoblastoma protein (Rb) to its target genes. Rb in turn recruits HDAC 1 which leads to transcriptional silencing of genes with important roles in tumor biology such as cyclin E (Brehm et al., Nature 391:597-601 (1998); Hake et al., Br J Cancer 90:761-769 (2004)). Aberrant modification of histones associated with DNA repetitive sequences has also been reported which include lower levels of H4K16ac and H4K20me3 in hematological malignancies and colorectal adenocarcinomas (Fraga et al., Nat Genet 37:391-400 (2005)). Furthermore, when examined at a global level by immunostaining of primary tumor tissues, individual tumor nuclei show variable levels of histone modifications, generating an additional layer of epigenetic heterogeneity at the cellular level (Seligson et al., Nature 435:1262-1266 (2005)). Thus, tumor cells may harbor aberrant patterns of histone modifications at individual promoters, repetitive elements and globally at the level of single nuclei.

In cancer patients, clinical outcome prediction is based generally on tumor burden and degree of spread with additional information provided by histological type and patient demographics. However, cancer patients with similar tumor characteristics still show heterogeneity in the course and outcome of disease. Therefore, accurate sub-classification of patients with similar clinical outcomes is required for development of targeted therapies and personalization of patient care (Ludwig, J. A., Weinstein, J. N., Nat Rev Cancer 5:845-856 (2005)). In this regard, molecular biomarkers have been useful in distinguishing subtypes of cancer patients with distinct clinical outcomes, thereby expanding our prognostic capabilities. Among the various biomarkers, expression analysis of genes, individually or especially in groups as molecular fingerprints(Golub et al., Science 286:531-537 (1999)), has been used widely to identify disease subtypes with differences in outcome in multiple cancers such as lymphomas (Alizadeh et al., Nature 403:503-511 (2000)) and breast cancers (Perouet al., Nature 406:747-752 (2000); Sorlie et al., Proc Natl Acad Sci USA 98:10869-10874 (2001); Sotiriou et al., Proc Natl Acad Sci USA 100:10393-10398 (2003)). Similar to gene expression, DNA methylation of specific genes have also been used as biomarkers, especially in predicting response to treatments (Esteller, M., Curr Opin Oncol 17:55-60 (2005)). For instance, in gliomas, methylation status of MGMT (O⁶-methylguanine-DNA methyltransferase) promoter region correlates with response or resistance to alkylating agents (Esteller et al., N Engl J Med 343:1350-1354 (2000)).

We showed previously that heterogeneity in cellular (i.e., global or bulk) levels of histone modifications can be detected by immunohistochemistry (IHC) at the level of whole nuclei of cancer cells in tissue specimens (Seligson et al., Nature 435:1262-1266 (2005)). In prostate cancer tissue from an individual patient, malignant cells exhibit dissimilar levels of histone modifications. The extent of dissimilarity in the levels of histone modifications—quantified as percent cell staining—differs between patients. These differences generate epigenetic patterns that, in the case of prostate cancer, predict risk of tumor recurrence after removal of the primary tumor. Of the five modifications that we examined in prostate cancer, H3K4me2 and H3K18ac proved to be the most informative of prognosis. The cellular patterns of these two modifications were sufficient to distinguish two groups of patients with distinct clinical outcomes, whom otherwise were not distinguishable by standard clinico-pathological variables (Seligson et al., Nature 435:1262-1266 (2005)). In general, patients with low cellular levels of H3K4me2 and H3K18ac (i.e., decreased percent cell staining) had poorer prognosis with significantly increased risk of tumor recurrence compared to patients with higher levels of the two modifications. These findings demonstrated a novel link between cellular epigenetic heterogeneity and clinical behavior in cancer patients.

Considering that histones and their modifications are present ubiquitously, our results in prostate cancer raised the possibility that histone modification patterns may serve as markers of prognosis in other cancer types. Furthermore, the prognostic utility of histone modifications may not be limited to the modifications examined so far. Other histone modifications may provide improved or complimentary prognostic capability. With respect to the gene expression prognosticators, expression of one or more genes can be predictive of clinical outcome, but in most cases the identity of prognosticator genes is different in different cancers. Extending this logic to epigenetics, one would expect that different histone modifications predict prognosis in different cancers. However, we provide evidence here that the lower cellular levels of the same two histone modifications that were most informative in prostate cancer, H3K4me2 and H3K18ac, distinguish patients with decreased survival probabilities in other adenocarcinomas (i.e., cancer of glandular epithelium), namely, cancers of lung and kidney. We did not examine the levels of the other three modifications from our original study⁷. However, we show that the cellular levels of another histone modification, H3K9me2, which is associated with gene activity and repression, is by itself a strong predictor of clinical outcome, with lower levels predicting poor outcomes in prostate and kidney cancers. Consistent with primary tissues, we show that prostate cancer cell lines also exhibit different cellular levels of histone modifications. These global differences in cancer cell lines are correlated with changes in histone modification levels at repetitive DNA elements and less so with promoter regions. Our findings suggest that the cellular levels of histone modifications may be general predictors of clinical outcome in adenocarcinomas of different tissue origins; and that global loss of histone modifications may be linked to a more aggressive cancer phenotype.

Sample collection and Tissue Microarrays (TMA). Following UCLA Institutional Review Board approval, formalin-fixed paraffin embedded specimens of benign and tumor tissues from human lung, kidney and prostate were obtained from the Department of Pathology from surgical cases occurring between 1984 and 2002. Sample collection was blinded to clinical data which were obtained after TMA construction. At least three tumor tissue core biopsies 0.6 mm in diameter were taken from selected morphologically representative regions of each paraffin-embedded sample and arrayed as described previously (Seligson, D. B., Biomarkers 10 Suppl 1:S77-82 (2005)). Tumor staging for all tissue types was performed according to the American Joint Committee on Cancer (AJCC) and the International Union Against Cancer (UICC) tumor-node-metastasis (TNM) classification of malignant tumors. T stage was determined from surgical pathology, N and M stages were determined by postoperative pathologic, clinical and/or radiographic data.

The study endpoint examined for lung and kidney cancers was disease specific death. The survival time, in months, was the period from disease diagnosis, or from surgery, to death (lung and kidney, respectively). Patients alive at last follow-up or those with deaths not due to disease were censored at last follow-up. Death of unknown cause was censored for lung cancers; all causes were known for kidney cancer patients. The endpoint for prostate cancers was disease recurrence, defined as a postoperative serum PSA of 0.2 ng/ml or greater. Patients without recurrence were censored at last follow-up. The Eastern Cooperative Oncology Group performance status (ECOG PS) was determined at initial presentation for kidney and lung cancers.

Lung cancer patients. The World Health Organization (WHO) histological classifications of carcinomas of the lung were used. The lung cancer TMA contained 285 patient samples of which 262 (92%) were clinically informative. 257 of 262 cases (98%) were also informative for H3K18ac and H3K4me2. Adenocarcinomas included tumors with bronchioloalveolar components. The lung tumors were graded according to AJCC Cancer Staging Manual. The median age of lung cancer patients in this cohort was 67 years (range 41-87) and the male to female ratio was 1:1.4. The median tumor size was 2.5 cm. The median follow-up in this cohort was 59.0 months (range 1.0-229 months).

Kidney cancer patients. Pathological tumor subtyping of kidney cancers was performed according to the 1997 UICC/AJCC classification of malignant tumors. Kidney tumors were taken from radical or partial nephrectomies of patients with renal cell carcinoma. Of the 379 cases on the TMA, 373 (98%) were clinically informative with a further 359 (96%) being informative for H3K18ac, H3K4me2 and H3K9me2. The median age of kidney cancer patients in the localized cohort was 63.5 (range 27-88) and the male to female ratio was 1.9:1. The median tumor size was 4.5 cm. The median follow-up in this cohort was 43.1 months (range 0.0-142 months).

Prostate cancer patients. Prostate cancers were all of the histological type “adenocarcinoma, conventional, not otherwise specified”. From 226 prostate cancer patients on the TMA who underwent radical retropubic prostatectomy, 212 were clinically informative, of which 185 (87%) were also informative for H3K9me2. Prostate grading was performed using the Gleason Score system (equivalent to Gleason Sum); “low grade” in our cohort included those cases of Gleason Score 2-6. The median age of prostate cancer patients in this cohort was 64 years (range 46-75). The median follow-up in this cohort was 60.0 months (range 2.0-120 months).

Immunohistochemistry (IHC) and Western blotting. A standard 2-step indirect IHC staining method was used for all antibodies as previously described (Seligson, D. B., Biomarkers 10 Suppl 1:S77-82 (2005)) using the DAKO Envision System. Primary rabbit anti-histone polyclonal antibodies were applied for 60 min at room temperature—for lung TMAs, H3K18ac (Suka et al., Mol Cell 8:473-479 (2001)) at 1:300 and H3K4me2 (Upstate) at 1:600 dilutions; for kidney TMA, H3K18ac at 1:400, H3K9me2 (Abcam) at 1:50 and H3K4me2 at 1:800 dilutions; for prostate TMA, H3K9me2 at 1:100; and for cell line IHC, H3K9me2 at 1:100 dilution from stock. The polyclonal rabbit anti-H3 (Abcam) was used at 5 μg/ml. Monoclonal anti-Ki-67 MIB-1 (7.5 μg/ml) and anti-human p53 DO-7 (15 μg/ml) (Dako) were used for Ki67 and p53 detection, respectively. Using a test TMA containing 20-40 cases, we optimized the concentration of each antibody to observe the greatest variation in the staining range within each tissue type. The sections were counterstained with Harris' Hematoxylin. Negative controls were identical array sections stained minus the primary antibody. For Western analysis, histones were acid-extracted from PC3 (bone metastasis of prostate cancer; ATCC) and LNCaP (lymph node metastasis of prostate cancer; ATCC) cell lines and subjected to standard western blotting.

Scoring of immunohistochemistry for all tissues. Semi-quantitative assessment of antibody staining on the TMAs was performed by pathologists blinded to all clinico-pathologic variables. Two pathologists scored all the TMAs but one per cancer set (lung TMA-V. M., kidney and prostate TMAs-H. Y.). We chose IHC and semi-quantitative analysis to generate the datasets because this is by and large the most common immunostaining method in clinical pathology settings, making our approach easily adoptable into current pathology laboratories. Only cancerous epithelial tissues were scored, and only primary tumor cells from the first surgery was included in the study. The lower acceptable limit for scoring a given tissue spot was 10 cells. However, in the majority of tumor spots there were between 100 and 1,000 cells, and for most cases the tumor was represented by more than one spot containing the target tissue (average marker-informative primary tumor tissue spots per case=3.1 for kidney, 2.4 for lung and 3.0 for prostate). Normal epithelium in cancer specimens, mesenchymal or infiltrating inflammatory cells and metastases were excluded from scoring. The frequency of positive nuclear expression (range 0-100%) was scored for each TMA spot using the ‘labeling index’ method. In order to produce a single representative staining for each case, the percent cell positivity from each tumor spot within each case was pooled and used to determine the percentile rank of patients in each dataset.

Statistical analysis. To test whether ordinal variables differed across groups, we used the Kruskal-Wallis test, a non-parametric multi-group comparison test. To visualize the survival distributions, we used Kaplan-Meier plots. A multivariate Cox proportional hazards model was used to test the statistical independence and significance of multiple predictors. The proportional hazard assumption was tested using scaled Schoenfeld residuals. To study whether the categorized histone expression groupings differed across patient strata, we used the Fisher's exact test. Log-rank tests were used to test the difference between survival distributions. A p-value <0.05 was considered significant.

Chromatin immunoprecipitation (ChIP) and microarray hybridization. ChIP was performed essentially as described (Wang et al., Mol Cell 17:683-694 (2005)). Briefly, formaldehyde was added for 10 min at 37° C. to growing cultures of cells. After PBS washing, cross-linked cells were scraped from the plates and washed with 1 ml of PBS containing protease inhibitors (Roche). Cells were lysed, incubated for 10 min on ice and immediately sonicated. 100 μl of the lysate were used for immunoprecipitation with anti-H3K9me2 or H3K18ac antibody; 10 μl of the lysate was used as input. After overnight reversal of crosslinking at 65° C., ChIPed and input samples were treated with RNase A for 30 min at 37° C. and subsequently purified using the Qiagen Qiaquick PCR purification Kit. 10 ng of each IP and INP DNA were amplified using the WGA Kit (Sigma). 2 μg of amplified material were labeled with Cy3 or Cy5 (PerkinElmer) using the Bioprime Labeling Kit (Invitrogen). DNA was mixed with 35 ml of random priming solution (Invitrogen Bioprime Kit) to a final volume of 75 boiled for 5 min and quickly cooled in an ice-water bath for 5 min The labeling reaction was completed with 60U Klenow, dNTPs (0.12 mM dATP, dGTP and dTTP and 0.06 mM dCTP), 1.28 mM Cy3 and Cy5 for input and ChIPed DNA, respectively, and incubated for 3 h at 37° C. The labeled DNA was purified using Qiagen Qiaquick PCR purification Kit and the incorporation was measured with Nanodrop. Hybridization onto the Human Promoter array (Agilent-G4489A), washing, and scanning were carried out according to the manufacturer's instructions. The arrays were scanned using an Agilent DNA Microarray scanner. Data extraction and analyses were carried out using the Agilent Feature Extraction software (version 9.1.3.1) and Chip Analytics software (version 1.2). Probe signals were normalized with Lowess normalization.

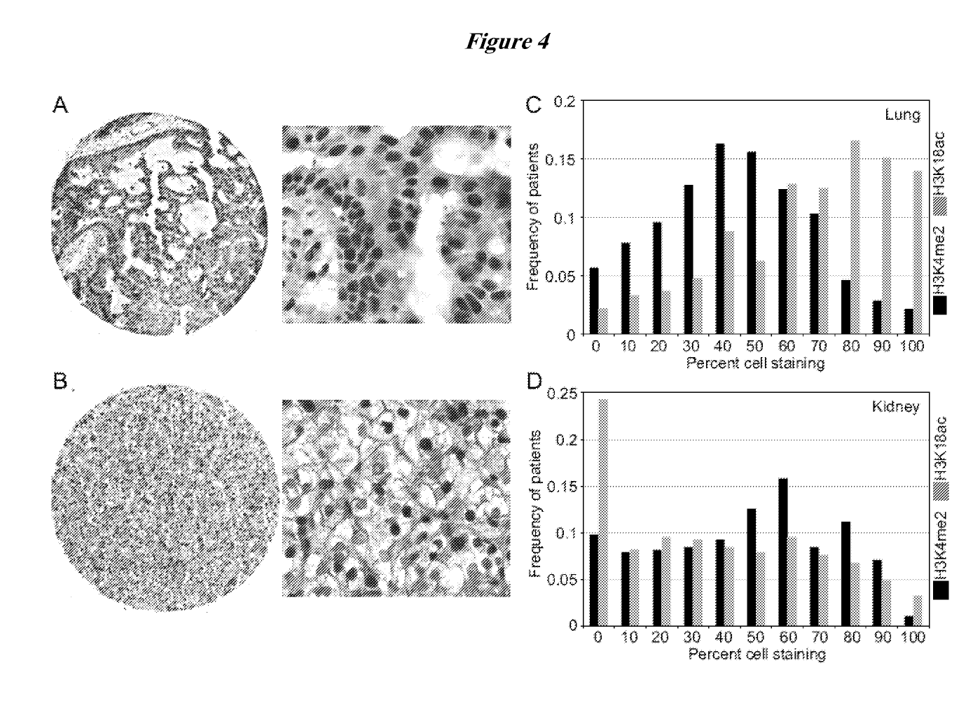

Detection of cellular histone modifications by immunostaining of cancer tissues. To determine the cellular levels of histone modifications in tissues obtained from patients, we combined IHC, a method for detecting the presence of specific antigens in cells, with Tissue Microarrays (TMA) (Seligson, D. B., Biomarkers 10 Suppl 1:S77-82 (2005); Kononen et al., Nat Med 4:844-847 (1998)), for high throughput analysis of a large number of tissue samples (Liu et al., J Biopharm Stat 14:671-685 (2004)). We analyzed the levels of H3K4me2, H3K9me2 and H3K18ac, using antibodies that recognize these specifically modified residues (Seligson et al., Nature 435:1262-1266 (2005); Suka et al., Mol Cell 8:473-479 (2001)), on TMAs of lung, kidney and prostate cancers. The choice of these cancers and the number of patients in each array were based on specimen availability with complete follow-up clinical data. Here, the global level of histone modifications refers to the percentage of cancer cells within each tissue sample that stained positively for a given antibody. This scoring system is used routinely and extensively for a wide range of biomarkers that are currently in clinical use in pathology laboratories. Shown in FIGS. 4A-B is representative cancer tissues from lung (FIG. 4A) and kidney (FIG. 4B) stained with anti-H3K18ac antibody (Objective: 10× left panel; 40× right panel). The cells with brown nuclei are considered positively stained, and their percentage within the tumor tissue is determined. The lack of staining by the histone modification antibodies is unlikely due to inaccessibility of their respective antigen as an anti-H3 antibody, which recognizes unmodified histone H3, stains positively in essentially all cells (data not shown). The unstained cells may still contain the modifications at certain genomic loci but their levels are below the detection limits of IHC, signifying that bulk histone modifications are considerably decreased in these cells.

Grouping of patients based on histone modification levels. In order to determine whether histone modifications predict clinical outcome, we first stratified patients into broad categories based on clinico-histological features such as grade or stage. The rationale for this initial stratification is that grade and stage are strong predictors of outcome (Ludwig, J. A., Weinstein, J. N., Nat Rev Cancer 5:845-856 (2005)). Grade is a histological measure of tumor differentiation. Stage is a measure of tumor size and spread beyond its original site. In general, higher grade and stage are associated with poorer outcome. However, within cancers that are of equivalent grade and stage, there are sub-types of patients that are molecularly heterogeneous and have different clinical outcomes (Ludwig, J. A., Weinstein, J. N., Nat Rev Cancer 5:845-856 (2005)). Prognostic biomarkers are therefore needed to sub-classify patients beyond grade and stage into more clinically cohesive groups. After grade or stage stratification, we assigned patients from each category into two groups according to a specific histone modification pattern or ‘histone pattern’ for short. This histone pattern was derived initially from an unsupervised clustering of prostate cancer patients, based on the cellular levels of H3K4me2 and H3K18ac staining that predicted clinical outcome. We did not search for new cut-off values for these two modifications in the current study. The histone pattern predicts that the patients with lower levels of H3K4me2 and H3K18ac have poorer prognosis than those with higher levels. After application of the histone pattern to patients in each cancer of lung and kidney, we tested the prediction that the two resulting groups should have significantly different clinical outcomes.

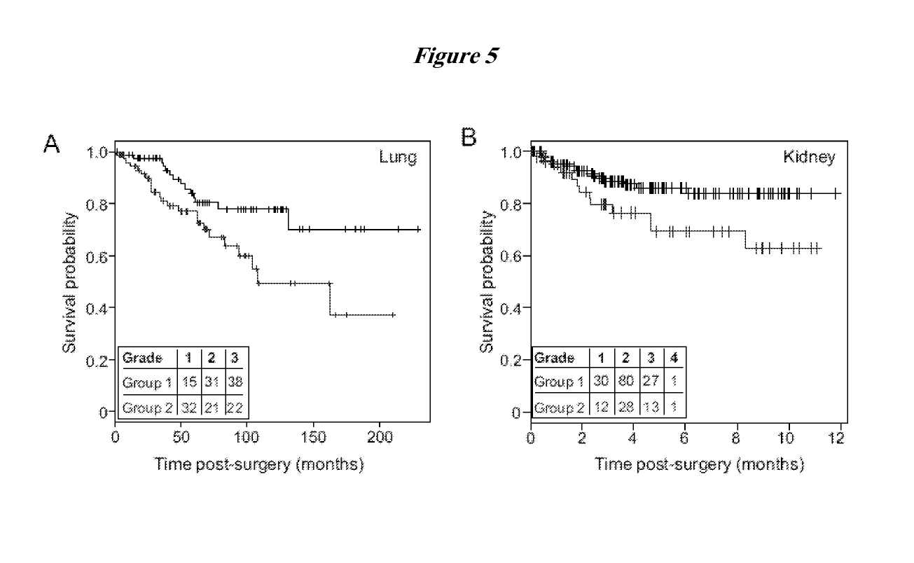

Histone modifications predict survival probability in lung cancer. To assess the distribution of staining for H3K4me2 and H3K18ac, we plotted the frequencies (y axis) of tissue samples in which the indicated percentage cell staining (x axis) were observed for each modification (FIG. 4C). H3K4me2 staining showed a broad distribution whereas H3K18ac staining was skewed toward higher percent cell staining (FIG. 4C). To determine whether histone modification patterns are clinically informative in lung cancer, we first partitioned the patients into stages 1 through 4 (data not shown). The patients were then assigned to two groups according to the predictive histone modification pattern that we identified from prostate cancer. The tumors with high levels of H3K4me2 and H3K18ac were assigned to Group 1 (i.e., H3K4me2>60 or H3K4me2 and H3K18ac>35 percentile staining); the remaining tumors with lower levels of the modifications were assigned to Group 2. In stage 1 lung adenocarcinoma (n=159), we found that the patients in Group 2 with lower cellular levels of histone modifications (red line, FIG. 5A) had a significantly lower 15-year survival probability compared to those in Group 1 (black line, FIG. 5A) (Log rank p=0.018, hazard ratio (HR)=2.19, 95%CI=1.13−4.27). Between the two groups, there was no difference in gender or age at surgery, but there was a statistically significant difference in grade distribution (p=0.0026). Paradoxically, the difference in grade distribution was due to presence of more low-grade tumors in Group 2 patients with poorer outcome (FIG. 5A inset box. In stages 2 (n=42), 3 (n=40) and 4 (n=16), we did not detect subgroups with significant differences in clinical outcome. Thus, the same prognostic histone modification pattern in prostate cancer serves as marker of prognosis in stage 1 lung adenocarcinoma.

The histone pattern is an independent prognosticator in lung cancer. To determine how the histone modifications compare to other known biomarkers in lung cancer, we examined the percentage of cells that stain positively for p53, which, when over-expressed, is associated significantly with poor patient outcome in stage 1 adenocarcinoma (Maddau et al., Am J Clin Pathol 125:425-431 (2006)). The expression levels of p53 were different in the two histone groups, with lower expression in the group with the poorer prognosis, 32.1% average positivity in Group 1 and 19.7% in Group 2 (p=0.033). So, the poorer prognosis predicted by the histone pattern is not due to increased incidence of p53 mutation. Additionally, in Groups 1 and 2, 30 and 25% of patients had a mitotic count >0, respectively (p=0.64), suggesting that the prognostication by the histone pattern is not due to increased proliferation rate. Finally, in a multivariate Cox model that included grade, mitotic count, p53, patients' performance status (ECOG), the histone groupings remained a significant predictor of outcome (Table 2). Thus, the histone modification patterns are independent predictors of clinical outcome in lung adenocarcinoma.

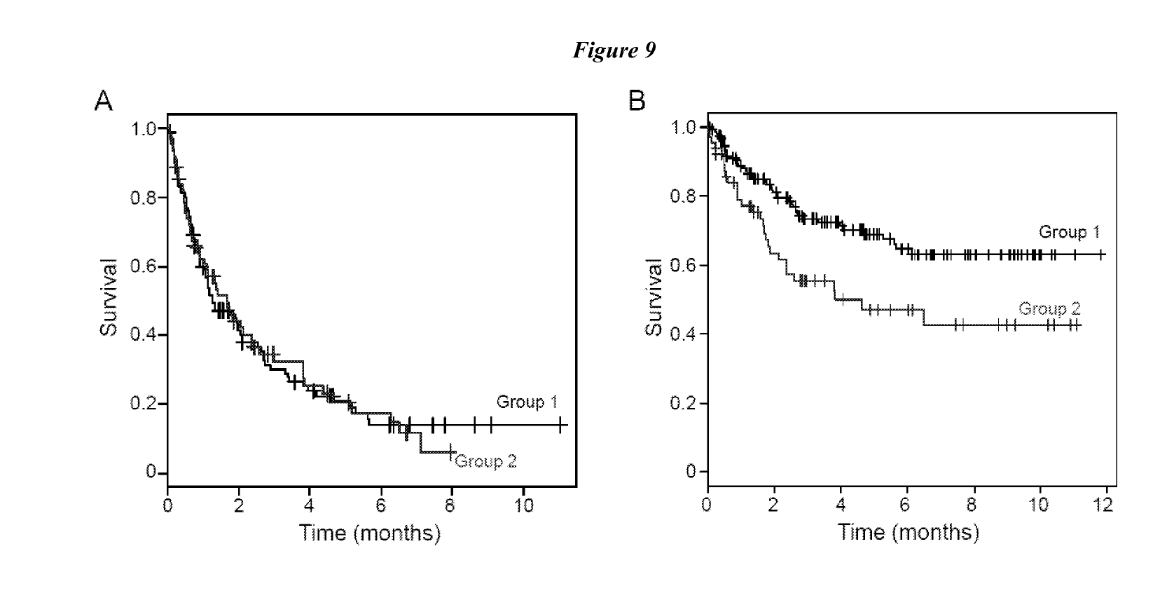

Histone modifications predict survival probability in kidney cancer. In kidney carcinoma, there was a broad distribution of staining levels for both H3K4me2 and H3K18ac with <10% of specimens showing 90-100% staining (FIG. 4D). Applying a similar histone pattern as above (i.e., >60 or >35 percentile staining for H3K4me2 and H3K18ac, respectively) to the patients with localized kidney tumors (n=192; data not shown), we identified two groups of patients which differ significantly in their survival probabilities (FIG. 5B). The patients with low levels of both modifications (Group 2) had a significantly poorer 1-year survival probability than those with higher levels of histone modifications (Group 1) (Log rank p=0.028, HR=2.22, 95%CI=1.07−4.62). There was no difference in the distribution of patients in the two groups according to gender, age at surgery, grade or stage (FIG. 5B inset box. In patients with metastatic disease (n=163), we did not detect subgroups with distinct clinical outcomes (see FIG. 9A). When patients were stratified only based on grade, the histone pattern distinguished two groups with significantly different survival probabilities in grades 1 and 2 but not in grades 3 and 4 cancers (data not shown). Thus, as in prostate and lung cancers, lower levels of the same two histone modifications predict poor clinical outcome in localized kidney adenocarcinoma.

The histone pattern is an independent prognosticator in kidney cancer. To determine how the histone modifications compare to other known biomarkers in kidney cancer, we examined the percentage of cells that stain positively for Ki67, a marker of proliferation, and p53. Increased expression of Ki67 or p53 was shown previously to be associated significantly with poor patient outcome in kidney adenocarcinoma (Shvarts et al., J Urol 173:725-728 (2005); Visapaa et al., Urology 61:845-850 (2003)). The median Ki67 expression levels were essentially the same in the two histone groups, 5% in Group 1 and 5% in Group 2 (p=0.50), indicating that the histone groupings are not due to their proliferation status. The expression levels of p53 were different in the two histone groups, with lower mean expression in the group with the poorer prognosis, 7.3% in Group 1 and 3.2% in Group 2 (p=0.0002). So, the poorer prognosis predicted by the histone modifications is not due to increased incidence of p53 mutation. In a multivariate Cox model that included grade, Ki67 and p53, the histone grouping remained a significant predictor of outcome (Table 2) but not when ECOG performance status was also included. Thus, the histone modification patterns are predictors of outcome in localized kidney cancer independently of grade, proliferation rate and p53 expression.

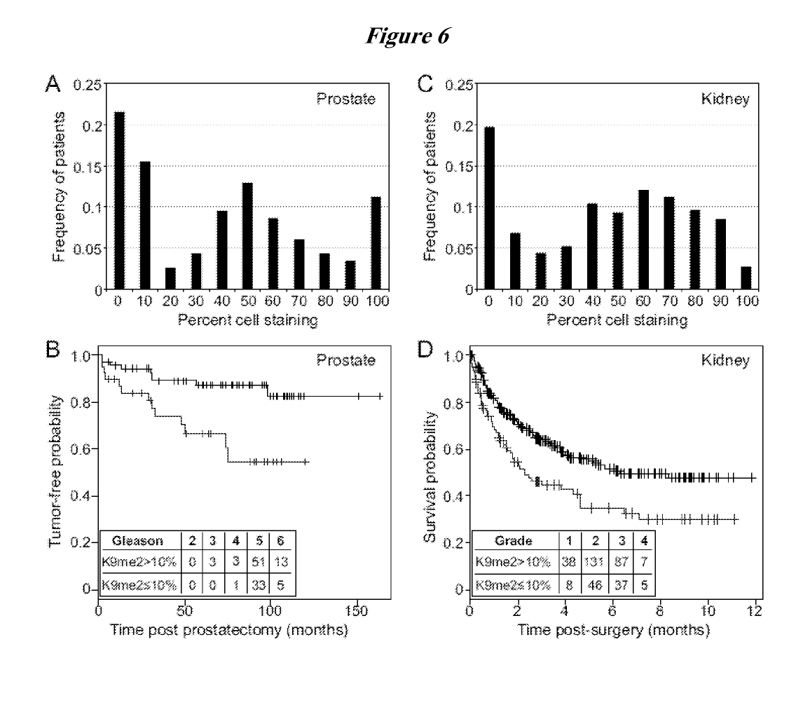

Cellular levels of H3K9me2 predict clinical outcome in prostate and kidney cancers. Both H3K4me2 and H3K18ac are modifications associated with gene activity. We next asked whether lower levels of H3K9me2—a modification associated with both gene repression and activity as well as heterochromatin—also predicts poorer prognosis in cancer. We determined H3K9me2 cellular levels in the same prostate and kidney cancer TMAs in which other modifications were examined. Distribution of staining in both prostate and kidney cancer specimens showed a broad pattern, ranging from 0 to 100% staining (FIGS. 6A and 6C). In prostate cancer, cellular levels of H3K9me2 were not predictive of outcome among patients with high Gleason score tumors (Score n=76). However, among the low Gleason score tumors (Score <7, n=109), the levels of H3K9me2 as a continuous, undichotomized variable was significantly related to tumor recurrence (Cox regression p=0.0037). Using Rpart tree, we then determined an optimal cut point in the levels of H3K9me2 to dichotomize patients into high and low levels of H3K9me2. As shown in FIG. 6B, patients with H3K9me2 staining (Group 2; red line) showed a higher risk of tumor recurrence compared with patients with >10% staining (Cox proportional hazard p=0.0043, HR=3.25, 95% CI 1.38-7.63). The prognostication by H3K9me2 was independent of tumor grade (FIG. 6B inset), stage, pre-operative PSA, and capsule invasion within the low Gleason score group (Table 2).

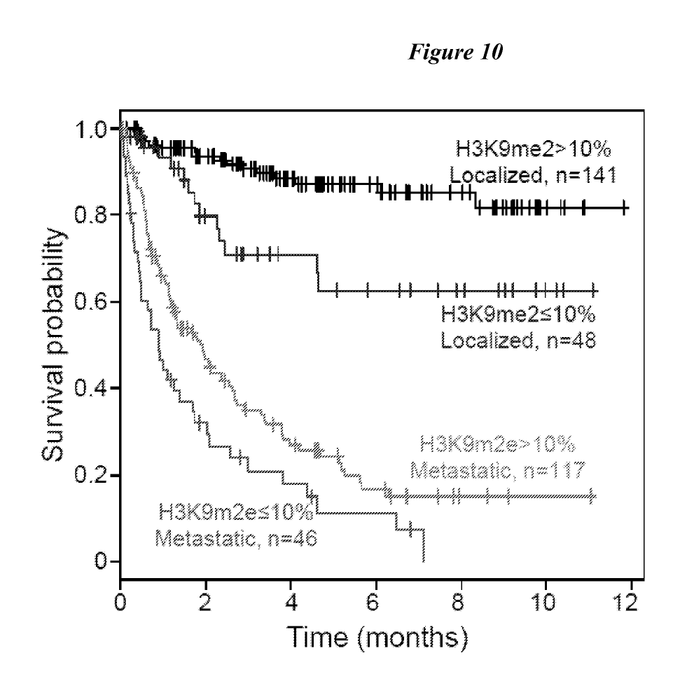

We next determined whether lower levels of H3K9me2 also predicts poorer prognosis in kidney cancer patients. Indeed the levels of H3K9me2 as a continuous, undichotomized variable was significantly related to survival probability in all kidney cancer patients (Cox regression p=0.028, n=359) and in patients with localized cancer (Cox regression p=0.026, n=189). Using the same cut point as in prostate cancer, kidney cancer patients with 0% H3K9me2 staining (Group 2; red line) showed significantly decreased survival probability compared with patients with >10% staining (Cox proportional hazard p=0.00092, HR=1.7, 95% CI 1.3-2.4; FIG. 6D). This was true for all patients and also within localized or metastatic disease strata (see FIG. 10. In a multivariate Cox model that included grade, Ki67, p53 and/or tumor localization, levels of H3K9me2 remained a significant predictor of outcome (Table 2). Taken together, our data indicate that lower cellular levels of H3K9me2 also predict poor prognosis in prostate and kidney cancers.

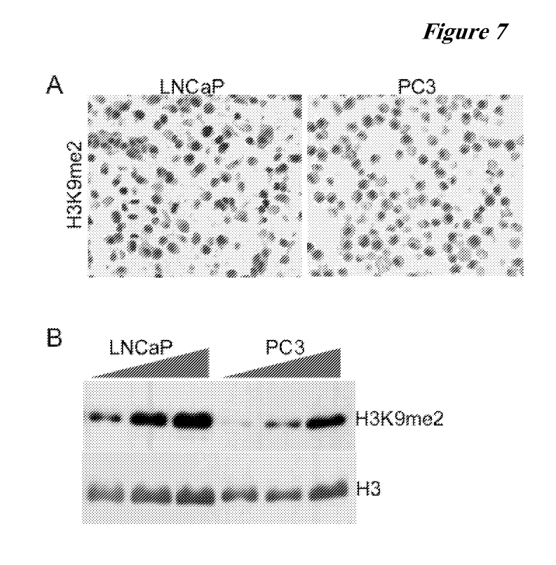

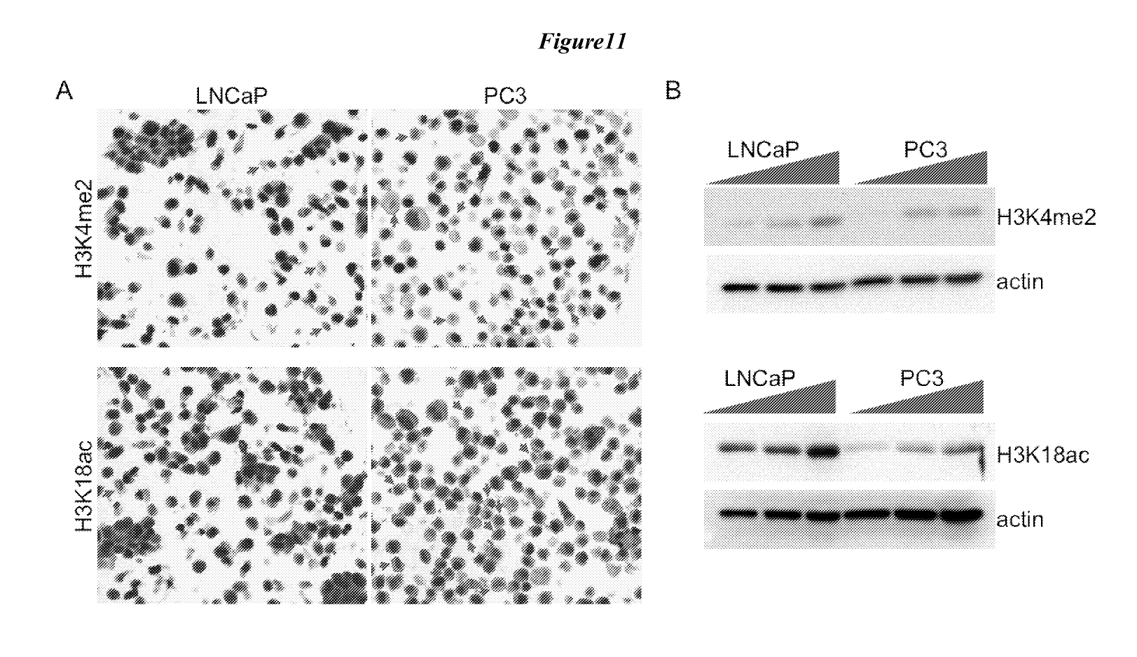

Changes in global levels of histone modifications correlate with their levels at repetitive DNA elements. To determine how cellular patterns of histone modifications map to individual promoters at the molecular level, we identified two prostate cancer cell lines that may serve as a model for observations in primary tumors. We expected the phenotypically more aggressive cancer cell line to contain generally lower levels of histone modifications. This was indeed the case for the LNCaP and PC3 prostate cancer cell lines. The PC3 cell line, derived from a bone metastasis of prostate cancer, is considered to be more aggressive than the LNCaP line which was isolated form a lymph node metastasis. FIG. 7A shows immunohistochemical staining of LNCaP and PC3 cells with an anti-H3K9me2 antibody. The more aggressive PC3 cells contained reduced H3K9me2 levels compared to LNCaP cells. Western blotting of acid-extracted histones confirmed the IHC results (FIG. 7B). PC3 cells also showed lower levels of H3K18ac and H3K4me2 compared to LNCaP cells (see FIG. 11).

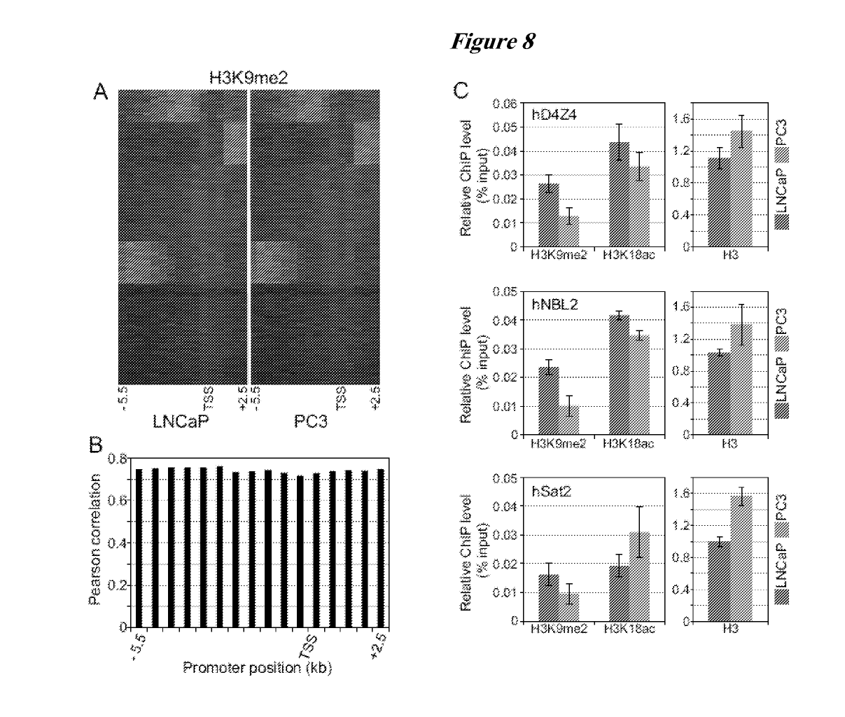

We next performed CUP-chip (chromatin immunoprecipitation combined with microarrays) experiments to compare the H3K9me2 distribution between LNCaP and PC3 cells at promoters genome wide (FIG. 8A). For each cell line, we compared the ChIPed DNA with an anti-H3K9me2 antibody to total genomic DNA (input). We used an Agilent Human Promoter Array containing 17,054 promoters, covering an average region from −5.5 kb to +2.5 kb with respect to the annotated transcription start site (TSS) of each promoter. The data for each gene was standardized to generate sixteen 500-bp fragments represented as columns in FIG. 8A. We found that distribution of H3K9me2 in LNCaP and PC3 cells were very similar with a high degree of correlation at each position across the promoters genomewide (FIG. 8B). So, the difference in total levels of H3K9me2 between LNCaP and PC3 cells is likely not due to global changes at gene promoters.

We next asked whether lower global levels of histone modifications in PC3 cells were due to decreased levels at repetitive DNA elements. These DNA elements, which collectively comprise ˜70% of the human genome, are significantly DNA demethylated and have lower levels of H4K16ac and H4K2Ome3 in certain cancers (Fraga et al., Nat Genet 37:391-400 (2005)). We used the same ChIPed DNA as above followed by quantitative real time PCR (qRT-PCR) to examine the levels of H3K9me2 at several DNA repetitive elements (FIG. 8C). To circumvent copy number variation, for each repetitive DNA elements, we examined the region at the boundary of repetitive and non-repetitive DNA elements. As shown in FIG. 8C, PC3 cells showed lower levels of H3K9me2 at subtelomeric repeat elements (D4Z4), a tandem 1.4-kb element found in acrocentric chromosomes (NBL2) and juxtacentromeric satellite 2 (Sat2) DNA sequences. Lower H3K9me2 levels were not due to histone loss (FIG. 8C). H3K18ac also showed lower levels at D4Z4 and NBL2 elements. These results indicate that global loss of histone modifications in more aggressive cancers correlate with lower levels of the modifications at DNA repetitive elements.

We have provided evidence that the global levels of the same histone modifications in cancer tissues predict disease outcome in different adenocarcinomas of lung and kidney in addition to the previously reported prostate cancer (Seligson et al., Nature 435:1262-1266 (2005)). Generally in each cancer, patients who have lower percentage of cancer cells that stain positively for H3K4me2 and H3K18ac have poorer prognosis than those with higher percentages. Interestingly, the cellular level of H3K9me2 is also associated with disease outcome, with lower levels predicting poorer prognosis in prostate and kidney cancers (we have not yet examined H3K9me2 in the lung cancer cohort). Thus, the general picture that emerges from our data is that the lower cellular levels of histone modifications are associated with poorer clinical outcome. Interestingly, the levels of histone modifications are correlated positively with each other, suggesting that loss of one histone modification is generally associated with loss of other modifications within a patient (see Table 3). Other laboratories have validated and extended the prognostic powers of histone modifications to yet other modifications and other cancers including non-small cell lung cancer (Barlesi et al., J Clin Oncol 25:4358-4364 (2007)) and cancers of breast, ovary and pancreas (Wei et al., Mol Carcinog 47:701-706 (2008)). This general applicability of histone modification patterns is unlike most prognostic markers described today. The prognostic power of the histone modifications is independent of clinico-pathological variables including proliferation rate as well as certain biomarkers such as p53 expression in lung and p53 and Ki67 expression in kidney cancers. Therefore, the cellular patterns of histone modifications add further non-redundant information to the current prognostic markers for prediction of clinical behaviour in cancer patients.

Analysis of histone modifications in cancer have typically focused on specific genomic loci such as individual gene promoters, revealing local perturbation of histone modifications with consequent effects on the expression of downstream genes. Extending this notion to the PC3 cells, which contain ˜50% less H3K9me2 compared to LNCaPs, we were surprised to find that ChIP-chip data from the two cell lines were essentially similar to each other. This suggests that differences in global levels of histone modifications are unlikely to arise from changes at gene promoters. However, ChIP analyses of three DNA repetitive elements showed decreased H3K9me2 levels in PC3 vs. LNCaP cells. Such correlations between global levels of histone modifications and their levels at repetitive elements, but not at gene promoters, were demonstrated previously for other cancers (Fraga et al., Nat Genet 37:391-400 (2005)). Since DNA repeat elements comprise ˜60-70% of genomic sequences (Li et al., Nature 409:847-849 (2001)), levels of histone modifications at these regions may account for the global differences observed in both cancer cell lines as well as in primary cancer tissues.

The repetitive elements are demethylated on DNA in cancer which may contribute to genomic instability (Feinberg, A. P., Tycko, B., Nat Rev Cancer, 4:143-153 (2004)). Our data and those of others (Fraga et al., Nat Genet 37:391-400 (2005)) now suggest that the repetitive elements may also get demethylated and/or deacetylated on their associated histones. The biological consequence of this ‘de-modification’ of histones at repetitive elements is unclear but is likely associated with a more aggressive phenotype because lower global levels of histone modifications predict poorer prognosis. The regulatory mechanisms that affect histone modifications at the repetitive elements are poorly understood but could be due to improper targeting, altered expression and/or activity of histone modifying enzymes through genetic mutations, expression changes and/or post-translational control (Esteller, M., Br J Cancer 94:179-183 (2006)). Since all histone modifications are reversible, increased activity of one set of histone modifiers, e.g. HDACs, could change the overall states of histone modifications to cause detectable changes at a global level (Kurdistani, S. K., Br J Cancer 97:1-5 (2007)). Some of these histone modifiers may preferentially affect DNA repetitive elements. Although this has not been demonstrated for mammalian proteins, the Hos3 HDAC in yeast preferentially deacetylates the ribosomal DNA repeats (Robyr et al., Cell 109:437-446 (2002)).

In potentially related studies, we have shown that viral oncoproteins, such as the adenovirus e1a, can alter global patterns of histone modifications in human cells through genomewide redistribution of specific histone modifiers away from most of the genome and restricting them to a limited but biologically related set of genes to favour cell replication and thus viral production (Ferrari et al., Science 321:1086-1088 (2008); Horwitz et al., Science 321:1084-1085 (2008)). As in the case of the e1a oncoprotein, loss of histone modifications at the DNA repetitive elements in primary cancers could also reflect redistribution of HATs and HMTs away from these regions and onto a smaller set of genes that confer an advantage to the cells in which this occurs. Whatever the mechanism, it remains to be determined whether the cells with little or no detectable histone modifications are derived from a single precursor cell (i.e., clonal) or from parallel loss of histone modifications in different tumor cells within a tissue.

The prognostication by the histone modifications might have implications for epigenetic therapy. One possibility is that the patients with poorer outcome who have low levels of H3K4me2, H3K18ac and/or H3K9me2 would benefit more from HDAC inhibitors than those with high levels of the histone modifications. It is also possible that poor outcome group would require a different regimen of various epigenetic therapeutics (Egger et al., Nature 429:457-463 (2004); Minucci et al., Nat Rev Cancer 6:38-51 (2006)). Whatever the case may be, the simplicity and robustness of our approach should facilitate the development of a standard and effective epigenetic assay to identify sub-sets of cancer patients with similar clinical outcome.

### Example 3

### Histone Modifications Predict Survival Probability in Breast Cancer

To determine whether histone modification patterns are clinically informative in breast cancer, we applied the histone pattern to patients with grades 1 and 2 breast tumors (n=33). The histone pattern identified two groups of patients with significantly different risks of tumor recurrence. The patients in Group 1 (i.e.,>60 or >35 percentile staining for H3K4me2 and K18ac, respectively) have <1% risk of 8-year tumor recurrence whereas those in Group 2 have 30% risk of recurrence (Log rank p=0.006). Between the two groups, there is no significant difference in grade, stage, estrogen and progesterone receptor status. Since mitotic index is one of three parameters that is incorporated into the breast cancer grading system, the histone groupings are also independent of mitotic index and thus proliferation rate. HER-2, a proto-oncogene whose overexpression is associated with poor outcome in breast cancer also showed no significant difference in the two histone groups: Her2 is over-expressed in 15/24 patients in Group land 2/9 in Group 2 (p=0.057). Thus, as in prostate, lung and kidney cancers, global levels of histone modifications serve as markers of prognosis in breast adenocarcinoma.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

TABLE 1
Multivariate Proportional Hazards Analyses: Overall and Disease-Free Survival for
RTOG 9704 Pancreatic Cancer TMA

| | Overall Survival | | | Disease-Free Survival | | |
|---|---|---|---|---|---|---|
| Histone Modification | Adjusted HR* | p- | Adjusted | Adjusted HR* | p- | Adjusted |
| Group | (95% CI) | value^{†} | for^{‡} | (95% CI) | value^{†} | for^{‡} |
| Low H3K4me2 | 1.51 (1.07-2.12) | 0.02 | 1, 2 | 1.38 (1.00-1.91) | 0.049 | 4 |
| Low H3K9me2 | 1.40 (1.01-1.94) | 0.04 | 1, 2, 3a, 3b | 1.74 (1.27-2.40) | 0.0007 | 4, 5 |
| Low H3K18ac | 1.35 (0.97-1.87) | 0.07 | 1, 2, 3a, 3b | 1.57 (1.13-2.18) | 0.007 | 3a, 3b, 4 |
| H3K4me2 and H3K18ac | 1.50 (1.05-2.14) | 0.03 | 1, 2 | 1.45 (1.03-2.02) | 0.03 | 2, 4 |
| (one or both low) | | | | | | |
| H3K4me2, H3K9me2 or | 1.56 (1.06-2.27) | 0.02 | 1, 2, 3b | 1.58 (1.11-2.27) | 0.01 | 4, 5 |
| H3K18ac (one or more low) | | | | | | |
| H3K4me2, H3K9me2 or | 0.52 (0.34-0.80) | 0.003 | 1 | 0.57 (0.37-0.87) | 0.009 | 4, 5 |
| H3K18ac (one or more high) | | | | | | |

*HR (Hazard Ratio): HR of 1 indicates no difference between the two groups of patients for the listed histone variable, while a HR > 1 indicates an increased risk of death/failure for histone group listed. With the exception of histone variable which was fixed, only variables that were kept in the model after backwards selection process are listed in the “adjusted for” column.
^{†}p-value from Chi-square test using the Cox proportional hazards model.
^{‡}The following covariates were used: 1. Nodal involvement (No vs. Yes), 2. KPS (90-100 vs. 60-80), 3a. Tumor Differentiation (Moderate vs. Well), 3b. Tumor Differentiation (Poor vs. Well), 4. Surgical Margins (Not unknown vs. Unknown), 5. T-Stage (T1/T2 vs. T3/T4)

TABLE 2
Multivariate proportional hazard analyses.

| | | | |
|---|---|---|---|
| Variable value | Hazard Ratio | 95% CI | p |

Histone pattern in stage 1 lung cancer (H3K4me2 and H3K18ac)

| | | | |
|---|---|---|---|
| ‘Histone pattern’ grouping | 4.94 | 1.67-14.63 | 4.0E−3 |
| Grade | 1.35 | 0.60-3.02 | 4.7E−1 |
| Mitotic count | 1.62 | 0.49-5.35 | 4.2E−1 |
| ECOG performance status | 2.00 | 0.74-5.42 | 1.7E−1 |
| p53 levels | 1.01 | 0.99-1.02 | 4.0E−1 |

Histone pattern in localized kidney cancer (H3K4me2 and H3K18ac)

| | | | |
|---|---|---|---|
| ‘Histone pattern’ grouping | 2.29 | 1.01-5.21 | 3.4E−2 |
| Grade | 2.20 | 1.25-3.88 | 6.4E−3 |
| Ki67 levels | 1.01 | 0.97-1.06 | 6.5E−1 |
| p53 levels | 1.03 | 1.01-1.05 | 1.6E−2 |

H3K9me2 in low grade prostate cancer

| | | | |
|---|---|---|---|
| H3K9me2 grouping | 2.95 | 1.08-8.00 | 3.4E−2 |
| Preoperative serum PSA (ng/ml) | 1.05 | 1.01-1.08 | 6.8E−3 |
| Grade | 1.21 | 0.37-3.92 | 7.5E−1 |
| Stage | 2.85 | 0.97-8.42 | 5.8E−2 |
| Capsule Invasion | 2.68 | 1.12-6.40 | 2.7E−2 |

H3K9me2 in all kidney cancer

| | | | |
|---|---|---|---|
| H3K9me2 grouping | 1.85 | 1.31-2.62 | 5.4E−4 |
| Tumor localization | 0.14 | 0.09-0.22 | <E−12 |
| Grade | 1.39 | 1.07-1.79 | 1.2E−2 |
| Ki67 levels | 1.01 | 1.00-1.03 | 1.6E−1 |
| p53 levels | 1.02 | 1.01-1.03 | 7.8E−5 |

H3K9me2 in localized kidney cancer

| | | | |
|---|---|---|---|
| H3K9me2 grouping | 2.26 | 1.03-4.92 | 4.1E−2 |
| Grade | 2.12 | 1.17-3.85 | 1.3E−2 |
| Ki67 levels | 1.01 | 0.97-1.06 | 6.2E−1 |
| p53 levels | 1.02 | 1.00-1.04 | 3.9E−1 |

TABLE 3
Cellular levels of histone modifications
are generally correlated with each other
Kidney Cancer-All Cases (n = 359):

| | H3K18ac | H3K4me2 | H3K9me2 |
|---|---|---|---|
| H3K18ac | 1 | | |
| H3K4me2 | 0.568 | 1 | |
| H3K9me1 | 0.618 | 0.730 | 1 |

Lung Cancer-All Cases (n = 257):

| | H3K18ac | H3K4me2 | H3K9me2 |
|---|---|---|---|
| H3K18ac | 1 | | |
| H3K4me2 | 0.546 | 1 | |
| H3K9me1 | NA | NA | 1 |

Prostate Cancer-All Cases (n = 188):

| | H3K18ac | H3K4me2 | H3K9me2 |
|---|---|---|---|
| H3K18ac | 1 | | |
| H3K4me2 | 0.688 | 1 | |
| H3K9me1 | 0.356 | 0.556 | 1 |

Shown in each table are case-level Pearson correlations across all clinical follow-up and histone marker-informative cases for the cancers indicated using mean percent positive cells.

1. A method of predicting the response of a cancer patient to therapy with 5-FU or another thymidylate synthase inhibitor, said method comprising determining the global histone modification level for H3K4me2, H3K9me2, or H3K18ac, or a combination thereof, in a cancer tissue sample from the patient.

2. The method of claim 1, wherein the presence of a low level of the histone modification indicates a poorer prognosis or likelihood for survival when treated with 5-FU or another thymidylate synthase inhibitor and the presence of a high global histone modification level for H3K4me2, H3K9me2, or H3K18ac indicates a better prognosis for survival when treated with 5-FU or another thymidylate synthase inhibitor wherein the cut-off between the high and low levels is based upon a statistical analysis of the global histone modification levels observed for a comparison group of thymidylate synthase inhibitor-treated cancer patients of known treatment survival or prognosis.

3. The method of claim 1, wherein the patient has node-negative cancer or is receiving 5-fluorouracil.

4. The method of claim 1, wherein positive tumor cell staining of the histone modifications H3K4me2, H3K9me2, or H3K18ac is used to classify the patient as low or high staining, wherein a low staining classification supports a prognosis of a poorer overall survival.

5. The method of claim 1, wherein the prognosis based upon histone modification levels of both H3K4me2 and H3K18ac, wherein a low histone modification level for both H3K4me2 and H3K18ac predicts a lower likelihood of survival.

6. The method of claim 4, wherein the histone modification levels are determined by immunocytochemistry or immunohistochemistry.

7. The method of claim 1, wherein the histone modifications levels for two or three of the histone modifications selected from H3K4me2, H3K9me2, and H3K18ac are used to provide the prognosis.

8. The method of claim 1, wherein the cancer is pancreatic cancer.

9. The method of claim 1, wherein the classification is based upon a histone rule.

10. The method of claim 1, wherein the cancer is an adenocarcinoma.

11. The method of claim 10, wherein the cancer is a low grade or low stage cancer.

12. The method of claim 1, wherein the cutoff dividing a lower from a higher level for the histone modification is about >30% for H3K9dime, about >60% for H3K4me2 or about >35 percentile staining H3K18ac.

13. A method of identifying a cancer patient for whom the additional administration of a histone deacetylase inhibitor to a 5-FU or other cancer therapy would be beneficial, comprising determining the level of the H3K18ac histone modification in a tissue sample from the pancreatic cancer of the patient, wherein a low level of the modification would indicate that the histone deacetylase inhibitor would be beneficial, wherein the cut-off between the high and low levels is based upon the H3K18ac global histone modification levels obtained for a comparison group of cancer patients of known survival or prognosis.

14. The method of claim 13, wherein 5-FU and the inhibitor are selected to treat the patient and the patient is so treated or so advised.

15. The method of claim 13, wherein the cancer is a low grade or low stage cancer.

16. A method of treating a patient having a pancreatic cancer, said method comprising
(a) contacting a cancer tissue sample from the patient with an antibody that specifically binds to a modified histone protein selected from the group consisting of H3K4me2, H3K9me2, and H3K18ac; and
(b) determining the levels of the modified histone protein in the tissue sample in comparison to levels observed for a comparison population(s) of known outcome; thereby providing a prognosis for said cancer; and
(c) administering a more aggressive anti-cancer therapy other than a thymidylate synthase inhibitor or in addition to the inhibitor when the prognosis indicates a cancer which is likely to have reduced survival or to be non-responsive to treatment with a thymidylate synthase inhibitor.

17. The method of claim 16, wherein the method predicts the likelihood of a recurrence of cancer.

18. The method of claim 16, wherein the thymidylate synthase inhibitor is 5-FU.

19. A method of identifying a cancer patient for whom therapy with gemcitabine or an agent which is not a thymidylate synthase inhibitor would be preferred over therapy with a thymidylate synthase inhibitor alone or with the thymidylate synthase inhibitor with leucovorin, comprising determining the level of the H3K18ac histone modification in a tissue sample from the cancer of the patient, wherein a low level of the modification as compared to a cut-off would indicate that the therapy with gemcitabine or the agent is preferred.

20. The method of claim 19, wherein the patient is administered gemcitabine.

21. The method of claim 19, wherein the thymidylate synthase inhibitor is 5-FU.

22. The method of claim 19, wherein the cancer is pancreatic cancer.

23. The method of claim 19, wherein the cut-off is based upon a statistical analysis of the global histone modification levels observed for a comparison group of thymidylate synthase inhibitor-treated cancer patients of known treatment survival or prognosis, wherein the cut-off demarcates the comparison group into two populations which differ by at least 20% in their response to therapy as judged by survival at 1 year.
